(19) United States
(12) Patent Application Publication
(10) Pub. No.: US 2024/0241133 A1
(43) Pub. Date: Jul. 18, 2024

(54) METHOD OF PROGNOSING PREECLAMPSIA

(71) Applicant: University of Tartu, Tartu (EE)

(72) Inventors: Ele Hanson, Tartu (EE); Kalle Kisand, Tartu (EE); Kristiina Rull, Tartu (EE); Maris Laan, Tartu (EE); Kaspar Ratnik, Tartu (EE)

(21) Appl. No.: 18/022,000

(22) PCT Filed: Aug. 16, 2021
(86) PCT No.: PCT/EP21/72661

§ 371 Date: Feb. 17, 2023

(30) Foreign Application Priority Data
Aug. 17, 2020 (GB) 2012830.2

(51) Int. Cl. G01N 33/68; C12Q 1/6844; C12Q 1/6883

(52) U.S. Cl. CPC G01N 33/689; C12Q 1/6844; C12Q 1/6883; C12Q 2600/118; G01N 2333/912; G01N 2333/96486; G01N 2800/368

(57) ABSTRACT

The present invention provides for a method of prognosing preeclampsia in a pregnant subject wherein the method comprises measuring the level of at least three biomarkers in a sample from the subject; optionally determining at least one clinical cofactor from the subject; generating a prediction value, wherein the prediction value indicates whether the subject will develop or will not develop preeclampsia during the ongoing pregnancy; the prediction value being based on the levels of the at least three biomarkers in the sample from the subject and optionally based on the at least one clinical cofactor.

### CROSS REFERENCE TO RELATED APPLICATIONS

The present application is a National Stage Application claiming priority from co-pending PCT Application No. PCT/EP2021/072661 filed Aug. 16, 2021, which in turn claims priority from Great Britain Application Serial No. 2012830.2 filed Aug. 17, 2020. Applicants claim the benefits of 35 U.S.C. § 120 as to the said PCT application, and priority under 35 U.S.C. § 119 as to the said Great Britain application, and the entire disclosures of all applications are incorporated herein by reference in their entireties.

### FIELD OF THE INVENTION

The present invention relates to a method of prognosing preeclampsia in a subject. The method comprises the measuring of biomarker levels in a sample taken from the subject, optionally determining at least one clinical cofactor from the subject, and generating a prediction value which indicates whether the subject will develop or will not develop preeclampsia within a time period during the pregnancy.

### BACKGROUND

Preeclampsia (PE) is a sudden and severe complication that develops during the second half of gestation and affects both the mother and the unborn child. Despite the general improvement of the socio-economic environment and availability of clinical service in developed countries over the past 20-30 years, the prevalence of PE has remained unchanged. Across 129 international independent studies, the overall estimates for preeclampsia and eclampsia were reported 4.6% (95% CI 2.7-8.2), and 1.4% (95% CI 1.0-2.0) of all deliveries, respectively (Abalos et al., 2013). In Europe, this extends up to 400,000 cases annually. Although the main clinical symptoms of PE are maternal hypertension and proteinuria, it may be accompanied with thrombocytopenia, renal and liver insufficiency, pulmonary oedema, cerebral and visual impairment and uteroplacental dysfunction. In extreme cases, the disease may progress to eclampsia characterized by severe eclamptic seizures due to cerebral oedema and cause maternal and fetal death. The challenge in the clinical management is to distinguish pregnancies with isolated gestational hypertension or increased protein levels in urine from the cases when these symptoms refer to the development of PE. Currently, the only truly curative intervention for PE is delivery of the baby. However, in a large proportion of PE cases it brings along premature birth (<37 gestational weeks) with a broad palette of complications to the new born.

PE is considered a ‘disease of placenta’, caused by impaired remodelling of spiral arteries during first half of pregnancy and/or suboptimal placental capacity to support the maternal-fetal needs until natural delivery. As the result of insufficient modulation of the uterine vasculature at the beginning of the pregnancy, hypertension and other signs of organ dysfunction, characteristic to preeclampsia, manifest during the second half of pregnancy. This is mediated by hypoxic placenta releasing biomolecules to maternal circulation that cause endothelial damage and generalized inflammatory vascular stress. At that point, however, it is too late to improve the function of sub-optimally developed placental vasculature. The only solution is to end the pregnancy and proceed with the delivery. The concept of shallow trophoblast invasion and inadequate spiral arteries remodelling is more typical for early PE, requiring delivery before 34 gestational weeks (g.w.). Late preeclampsia (delivery at or after 34 g.w.) might be caused by relative ischemia when placental growth reaches its limits at the end of pregnancy or due to a large fetus.

Despite the lack of targeted cure, it is acknowledged that early identification of pregnancies that are susceptible to PE would enable timely personalized clinical management and reduction of the most severe consequences of PE. Currently, initial assessment of a potential risk to develop PE is based on maternal increased blood pressure and/or presence of protein in the urine. Medical history of existing chronic hypertension, nulliparity and other conditions linked with increased risk to PE are also considered. However, this approach lacks sensitivity and specificity as only 35-40% of all cases of PE show any of these pre-existing risk factors. Further, assessment of maternal risk factors only has a detection rate of 30.4% for PE during the ongoing pregnancy and 40.8% for PE before 34 gestational weeks (early-onset of PE, EO-PE). The remaining challenge in the clinical management is to distinguish pregnancies with isolated gestational hypertension or increased protein levels in urine from the cases when these symptoms refer to the developing PE.

One of the most widely used and validated algorithm for detecting PE, published by The Fetal Medicine Foundation, London, UK (https://fetalmedicine.org/research/assess/preeclampsia/first-trimester) is based on combining maternal factors, uterine arteries pulsatility index, mean arterial pressure (MAP), placental growth factor (PIGF) and optionally placenta associated protein A (PAPP-A) (O'Gorman, 2017). For preterm PE, this screening test results in a detection rate of 75%, 89% for PE onset <32 gestational weeks and <50% for term PE (Table 1). However, established tests have several shortcomings:
Most currently developed algorithms include measurement of uterine artery pulsatility index (UtA PI) that is not a routine procedure in pregnant women's clinics in many countries. UtA PI performance also needs certified apparatus and certified qualified specialists.
The developed algorithms require that clinical biomarkers UtA PI and MAP, as well as blood draw for the serum biomarker measurements, are assessed on the same gestational day. In many centres, this is not practically possible.
For the currently implemented screening tests, serum biomarkers are measured individually and data for other parameters are combined for estimating the PE risk using the developed algorithms. This may be time-consuming, costly and increase the risk for technical errors.
For many biomarkers, including the most broadly applied markers PAPP-A and PIGF, the measurements also depend on the equipment and reagents used at a specific laboratory and for PE risk, estimation is based on the multiple of the median values of the specific laboratory. This reduces the broad applicability, precision, transferability and comparability of the proposed screening test in clinical centres with variable settings in different countries.
Several reported algorithms combining multiple biomarkers fail to prove the effectiveness in multiple populations (reviewed by Mosimann 2020). This may be explained by their non-linear dynamics and constant mutual modulatory interactions.
Not all biomarkers included in the prediction models are sufficiently specific to PE (e.g. low PAPP-A may also refer to fetal chromosomal abnormalities, risk of fetal death, growth restriction or preterm birth; low PIGF reflects, in general, placental insufficiency)

Dependable and early prediction is therefore crucial for successful treatment interventions in hypertensive disorders of pregnancy including, inter alia, PE. Consequently, provision of further, alternative and preferably improved methods and means for prediction and/or prognosis of hypertensive disorders of pregnancy continues to be of prime importance. There is therefore a need to provide a robust, cost-effective test to prognose PE in expectant mothers.

### SUMMARY OF THE INVENTION

The Inventors have surprisingly found that the method of the present invention allows for a precise and usable prognosis of preeclampsia (PE), i.e. whether the subject will develop or will not develop PE during the ongoing pregnancy. In particular, such precision and usability enables accurate early prediction of PE allowing the application of interventions to minimise the worst consequences of PE. The exclusion or ruling out of the development of PE will minimise any further follow up and reduce maternal stress during pregnancy.

Thus, in a first aspect of the present invention, there is provided a method of prognosing preeclampsia in a subject wherein the method comprises, measuring the level of at least three biomarkers in a sample from the subject;

optionally determining at least one clinical cofactor from the subject;

generating a prediction value, wherein the prediction value indicates whether the subject will develop or will not develop preeclampsia during the ongoing pregnancy;

the prediction value being based on the levels of the at least three biomarkers in the sample from the subject and optionally based on the at least one clinical cofactor; wherein the at least three biomarkers are PTX3, sFlt1, ADAM12 and optionally sENG; or PTX3, ADAM12, sENG and optionally sFlt1;

the at least one optional clinical cofactor selected from gestational age, parity e.g. nulliparity or multiparity, and the placental or fetal genotype of the rs4769613 T/C single nucleotide polymorphism e.g. TT-, CC- or TC- genotype; wherein a high PTX3, high ADAM12 and a high sFlt1 level, and if included in the calculation of the prediction value, a high sENG, as compared with control indicates an increased probability of the subject developing preeclampsia; or a high PTX3, high ADAM12 and a high sENG level, and if included in the calculation of the prediction value, a high sFlt1, as compared with control indicates an increased probability of the subject developing preeclampsia; and if included in the calculation of prediction value, a low gestational age, nulliparity, placental or fetal TC- and CC-genotypes of the rs4769613 T/C single nucleotide polymorphism indicates an increased probability of the subject to developing preeclampsia.

Advantageously, it is expected that the method may be used to prognose, i.e. whether the subject will develop PE or will not develop PE in pregnant women at or after the 231st gestational day. The method comprises measuring the level of at least three biomarkers in a sample, e.g. blood sample, from the subject preferably collected at or after gestational week 10+0 (day 70) and before gestational week 14+1 (day 99), preferably the at least three biomarkers are selected from PTX3, sFlt1 and ADAM12 or PTX3, ADAM12 and sENG. In at least some embodiments, at least one clinical cofactor from the subject is also determined, preferably the at least one clinical cofactor is selected from gestational age, parity e.g.

nulliparity or multiparity, and the placental or fetal genotype of the rs4769613 T/C single nucleotide polymorphism e.g. TT-, CC- or TC- genotype.

In some embodiments of the invention, the biomarkers are PTX3, sFlt1 and ADAM12, or PTX3, ADAM12 and sENG, or PTX3, sFlt1, ADAM12 and sENG and the clinical cofactors at blood sampling are gestational age and parity, or gestational age and the placental or fetal genotype of the rs4769613 T/C single nucleotide polymorphism, or gestational age, parity and the placental or fetal genotype of the rs4769613 T/C single nucleotide polymorphism.

The prediction value may, for example, be calculated using the following formulas:

$$p ( i ) = 1 / ( 1 + {e}^{∧} ( - a - b \times log 2 ( [ PTX 3 ] ) - c \times log 2 ( [ sFlt 1 ] ) - d \times log 2 ( [ sENG ] ) - e \times log 2 ( [ ADAM 12 ] ) - f \times parity - g \times gest . age - h \times placental or fetal genotype of rs 4769613 SNP ) )$$

$$or$$

$$p ( i ) = 1 / ( 1 + {e}^{∧} ( - a - b \times log 2 ( [ PTX 3 ] ) - c \times log 2 ( [ sFlt 1 ] ) - d \times log 2 ( [ sENG ] ) - e \times log 2 ( [ ADAM 12 ] ) - f \times parity - g \times gest . age ) )$$

$$or$$

$$p ( i ) = 1 / ( 1 + {e}^{∧} ( - a - b \times log 2 ( [ PTX 3 ] ) - c \times log 2 ( [ sFlt 1 ] ) - d \times log 2 ( [ sENG ] ) - e \times log 2 ( [ ADAM 12 ] ) - g \times gest . age - h \times placental or fetal genotype of rs 4769613 SNP ) )$$

A prediction value (p(i)) equal to or higher or greater than the threshold value, indicates that the subject will develop preeclampsia at or after gestational week 33+0 (day 231) until delivery, i.e. is predictive of PE.

A prediction value (p(i)) lower or less than a threshold value, indicates that the subject will not develop preeclampsia at or after gestational week 33+0 (day 231) until delivery, i.e. excludes the risk of developing PE.

### BRIEF DESCRIPTION OF THE FIGURES

FIG. 1 shows distribution of maternal serum biomarkers measured with Luminex® 6PLEX assay during 10+0 until 14+0 (inclusive) gestational weeks, stratified by the onset of preeclampsia (PE) during the ongoing/index pregnancy (n=14) vs uncomplicated gestations until term (n=20). Whiskers on the plot show median with interquartile range. Statistical difference in biomarker distributions between PE and control cases was compared using Mann-Whitney U-test.

FIG. 2 shows Spearman's rank correlation between sENG and sFlt-1 measurements of sera drawn from pregnant women within 70 and 98 gestational days. Grey around the linear regression line (y=0,7651x+1931.4) indicates the 95% confidence region.

FIG. 3 shows the characterization of the area under curve (AUC) for the best performing 1st trimester preeclampsia (PE) prognosis models 4A (serum PTX3, ADAM12 and sFlt-1 adjusted for gestational age and parity), 5C (serum PTX3, ADAM12, sFlt-1 and sENG adjusted for gestational age combined with the placental or fetal genotype of the SNP rs4769613 T/C as a cofactor) and 6C (serum PTX3, ADAM12, sFlt-1 and sENG adjusted for gestational age combined with the placental or fetal genotype of the SNP s4769613 T/C and parity as cofactors). Parity was treated as binary marker, whereby a pregnant woman was assigned as nulliparous, referring to no previous deliveries or multiparous, referring to at least one childbirth before the ongoing/index pregnancy. Sensitivity and specificity values are provided in Table 4 and coefficients for the PE prognosis formulae in Table 6. The abbreviation AUC refers to Area Under Curve.

FIG. 4 shows the distribution of maternal serum biomarkers measured with Luminex® 6PLEX assay during 10+0 until 14+0 g. weeks (inclusive), stratified by parity. Nulliparity refers to no previous deliveries, whereas multiparity refers to women with at least one childbirth before the ongoing/index pregnancy. Whiskers on the plot show median with interquartile range. Statistical difference in biomarker distributions between nulliparous (n=21) and multiparous cases (n=13) was compared using Mann-Whitney U-test.

### DETAILED DESCRIPTION OF THE INVENTION

It is to be understood that different applications of the disclosed methods may be tailored to the specific needs in the art. It is also to be understood that the terminology used herein is for the purpose of describing particular embodiments of the invention only and is not intended to be limiting.

In addition, as used in this specification and the appended claims, the singular forms “a”, “an”, and “the” include plural referents unless the content clearly dictates otherwise. Thus, for example, reference to “an inhibitor” includes two or more such inhibitors, or reference to “an oligonucleotide” includes two or more such oligonucleotide and the like.

The term “biomarker” is widespread in the art and may broadly denote a biological molecule and/or a detectable portion thereof whose qualitative and/or quantitative evaluation in a subject is, alone or combined with other data, predictive and/or informative (e.g., predictive, and/or prognostic) with respect to one or more aspects of the subject's phenotype and/or genotype, such as, for example, with respect to the status of the subject as to a given disease or condition. Particularly, biomarkers may be metabolite-, RNA- (esp. mRNA-), peptide-, polypeptide- or protein-based, preferably peptide-, polypeptide- or protein-based.

The terms “gestational age”, “age of gestation” and similar are widespread in the art and commonly denote the time as measured in days or weeks, preferably days, from the 1st day of a female's last menstrual period. Thus, gestational age may be defined as the age of the pregnancy from the last normal menstrual period. Normal term pregnancy may be defined as a length of 259 gestational days and longer (up to 294 days), preferably 280 days. A human pregnancy of normal gestation may alternatively be defined as between about 38 and 42 weeks, preferably about 40 weeks. References to X+Y gestational weeks means X weeks plus Y days, e.g. 14+1 gestational weeks means 14 weeks plus 1 day.

The terms “fetal” and “placenta” represent any tissues, cells or DNA of fetal origin, including amniotic cells and DNA in amniotic fluid; biological material or DNA derived from any structures of the fetal side of the placenta, e.g. chorionic villi or placental cells/DNA circulating in maternal bloodstream; cells/DNA derived from an embryo generated by in vitro methods applied in assisted reproductive technologies (ART).

The term “fetus” may also be referred to inter alia as “conceptus” or “embryo”.

All publications, patents and patent applications cited herein, whether supra or infra, are hereby incorporated by reference in their entirety.

### Prognosing Preeclampsia

The method of the invention has utility in a number of applications including prognosing preeclampsia (PE), predicting whether a subject, specifically a pregnant subject, will develop PE or whether PE risk can be excluded. Preferably, the method of invention is applied at or after 10+0 and before 14+1 gestational weeks. Preferably, the method of the invention prognoses PE. The method of the invention may also have utility in choosing optimal management of preeclampsia in a subject and/or monitoring the effect of a possible treatment or treatments. Preferably, the method of the invention has utility in prognosing PE, predicting whether a subject, in particular a pregnant subject, will or will not develop PE at the beginning, start of, or during the third trimester of pregnancy. Preferably the method of the invention has utility in prognosing PE in a pregnant subject by predicting whether the pregnant subject will or will not develop PE at or after gestational week 33+0 (day 231) until delivery. The method of the invention facilities determining an appropriate treatment regimen for the subject.

The term “preeclampsia” or “pre-eclampsia” means a multisystem complication of pregnancy comprising high blood pressure or hypertension and one or more additional symptoms selected from: swelling of the hands, feet and/or face (oedema), persisting headache, visual disturbances, liver, renal or hematologic dysfunction (including proteinuria, elevated liver enzymes, hemolysis, thrombocytopenia), epigastric pain and eclamptic seizures and uteroplacental dysfunction. PE generally denotes a pregnancy-associated disease or condition that resolves by 12 weeks postpartum.

Based on the most recent recommendations by the International Society for the Study of Hypertension in Pregnancy (ISSHP) (Brown et al. 2018) preeclampsia can be diagnosed when gestational hypertension is accompanied by >1 of the following new-onset conditions at or after 20 weeks' gestation:
Proteinuria: protein 1+by dipstick on urine analysis, >300 mg of protein in a 24-hour urine collection, or a single random urine sample having a protein/creatinine ratio >0.3 mg/mg
Other maternal organ dysfunction, including: acute kidney injury, liver involvement with or without right upper quadrant or epigastric abdominal pain, neurological or haematological complications
Uteroplacental dysfunction: such as fetal growth restriction, abnormal umbilical artery Doppler wave form analysis, or stillbirth

Gestational hypertension is defined as a systolic blood pressure (BP)>140 mmHg and/or a diastolic BP >90 mmHg after 20 weeks' gestation (generally measured on two occasions over 4 hours apart, e.g. about 4 to about 100 hours apart).

PE typically occurs in the third trimester of pregnancy, i.e. at or after 28th week of pregnancy. PE may already occur at or after the 20th week of pregnancy. If preeclampsia is not treated, it can lead to brain oedema that causes eclamptic seizures that are not related to a pre-existing neurological condition, and even death of the mother and/or baby.

Prognosing PE or PE prognosis means providing a prediction of whether a subject will or is likely to develop PE. Prognosing PE or PE prognosis is a PE prediction or prediction of PE onset. Prognosing PE is a prediction of the subject's susceptibility or risk of developing PE;

a prediction of the course of disease progression and/or disease outcome, for example expected onset of the PE, expected severity and course of the PE, expectations as to whether the PE will develop into eclampsia; a prediction of the subject's responsiveness to treatment for the PE, for example, a prediction of a subject's responsiveness to treatment for the PE, for example positive response, a negative response, no response at all. Prognosis includes predicting whether or not an individual will develop PE, whether or not they will need treatment and/or whether the progress of the disease will be fast or slow. In an embodiment of the present invention, a prediction value is generated wherein the prediction value indicates whether the subject will develop or will not develop PE, or has PE. Monitoring preeclampsia means monitoring a subject's condition, for example to inform a preeclampsia prognosis and/or to provide information as to the effect or efficacy of a preeclampsia treatment. Treating preeclampsia means prescribing or providing treatment of preeclampsia in a woman and may include preventing the preeclampsia from occurring in a subject which may be predisposed to preeclampsia; inhibiting preeclampsia, i.e. arresting preeclampsia development; relieving, curing or regressing preeclampsia. If no preeclampsia is predicted in the subject, no special monitoring of the subject is required for at least two months. If preeclampsia is predicted, the subject has to be monitored regularly, for example appointments with the subject each week to measure blood pressure; to test urine protein level; documenting excessive weight gain; monitoring of fetal wellbeing; measuring blood liver enzymes; hemogram; coagulogram; headache frequency; visual disturbances; epigastric pain, swelling of the hands or face.

### Subject and Sample

The terms “individual,” “subject,” “host,” and “patient,” are used interchangeably herein and refer to any subject for whom prognosis, treatment, monitoring or therapy is desired. An individual, subject, host or patient may be a human. The individual, subject, host or patient is preferably a human. Preferably the individual, subject, host or patient is a pregnant individual, subject, host or patient, more specifically a pregnant human subject. The subject may be asymptomatic for preeclampsia.

The term “biological sample” encompasses a variety of sample types obtained from an organism and can be used in a prognostic or monitoring assay. The biological sample may be any sample derived from the subject. Samples may include, without limitation, whole blood, plasma, serum, red blood cells, white blood cells (e.g., peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, nail clippings, cell lysates, cellular secretion products, inflammation fluid, vaginal secretions, or biopsies such as preferably placental biopsies. Preferred samples may include ones comprising any one or more biomarkers as taught herein in detectable quantities. The biological sample may be a blood sample or other liquid samples of biological origin. Biological sample may refer to samples that have been manipulated in any way after their procurement, such as by treatment with reagents, solubilisation, or enrichment for certain components. The term encompasses a clinical sample and also serum, plasma, biological fluids and tissue samples. Preferably, the sample is a blood sample, for example whole blood, serum or plasma. The sample may be a blood serum sample.

The sample, e.g. blood sample, is typically obtained from the individual any time after confirmation of pregnancy, preferably during the first trimester of gestation. Gestation means the duration of pregnancy in a human, i.e. the time interval of development from fertilisation until birth, plus two weeks, i.e. to the first day of the last menstrual period. Thus, gestational age is defined as the age of the pregnancy from the last menstrual period. The first, second or third trimester means the first, second or third portions of gestation, each segment being three months long. Thus, for example, the first trimester means from the first day of the last menstrual period through the 13th week of gestation; the second trimester means from the 14th through to the 27th week of gestation; the third trimester means from the 28th week through to birth, i.e. 38-42 weeks of gestation. A sample, e.g. blood sample, may be obtained at about weeks 10 through to 14+0 of gestation, at about weeks 11 through to 14+0 of gestation, at about weeks 12 through to 14+0 of gestation, at about weeks 13 through to 14+0 of gestation, at about weeks 14. The sample, e.g. blood sample, may be collected at any point between the 10th and 14th gestational week during the first trimester.

Thus, in some embodiments, the subject sample, e.g. blood sample, may be obtained early in gestation, e.g. at week 10 or more of gestation, e.g. at week 11, 12, 13 or 14 of gestation. Preferably the sample, e.g. blood sample, is collected at or after gestational week 10+0 (70th gestational day) and before gestational week 14+1 (99th gestational day). The sample, for example the blood sample, may be collected at or after the 70th, 75th, 80th, 85th, 90th, 95th, 97th or 98th gestational day and before the 99th gestational day. In one embodiment of the present invention, the sample, e.g. blood sample, is collected at or after the 70th gestational day and at or after the 98th gestational day. In one embodiment of the invention, the preeclampsia prognosis is made immediately after the sample, e.g. blood sample is collected. Alternatively, the preeclampsia prognosis is made at any time during the pregnancy after the sample, e.g. blood sample, is collected, e.g. 24, 48, 72 or 96 hours after the sample, e.g. the blood sample, is collected. The preeclampsia prognosis may predict whether the subject will or will not develop preeclampsia at or after gestational week 33+0, i.e. the 231st gestational day until delivery. The preeclampsia prognosis may predict whether the subject will or will not develop preeclampsia during the second half of pregnancy, i.e. from 20 gestational weeks onwards. The preeclampsia prognosis may predict whether the subject will or will not develop preeclampsia at or after gestational week 20 until delivery.

The preeclampsia prognosis may predict whether the subject will or will not develop preeclampsia at or after gestational week 20 and at or before gestational week 33+0. The preeclampsia prognosis may predict whether the subject will or will not develop preeclampsia at or after gestational week 20, 21, 22, 23, 24, 25, 26, 27, 28, 29, 30, 31 32, 33, 34, 35, 36, 37, 38, 39, 40, 41 or 42.

Once a sample, e.g. blood sample, is obtained, it can be used directly; or frozen and used after thawing at any time later. Samples may include samples derived from humans.

### Biomarkers

Biomarkers or preeclampsia biomarkers are molecular entities whose representation or determination in a sample, for example a blood sample, is associated with a preeclampsia phenotype. Biomarker concentrations follow tight gestational dynamics, i.e. the normal expected range of concentrations of a specific biomarker is dependent on the gestational age. For example, the concentration of a biomarker in a sample, e.g. blood sample, may be high in early pregnancy, but decrease or drop in the late pregnancy or vice versa, the concentration of biomarker in a sample, e.g. blood sample, may be low in early pregnancy, but increase in the late pregnancy.

Biomarkers may be differentially represented, i.e. represented at a different level, in a sample, e.g. blood sample, from an individual that will develop or has developed preeclampsia as compared to a healthy individual. In some instances, an elevated level of biomarker at the gestational age of sampling is associated with the preeclampsia phenotype. For example, the concentration of biomarker in a sample, e.g. blood sample, may be 1.5-fold, 2-fold, 2.5-fold, 3-fold, 4-fold, 5-fold, 7.5-fold, 10-fold, or greater more concentrated in a sample, e.g. blood sample, associated with the preeclampsia phenotype than in a sample, e.g. blood sample, not associated with the preeclampsia phenotype or the concentration of marker in a sample, e.g. blood sample, may be 10%, 20%, 30%, 40%, 50% or greater more concentrated in a sample, e.g. blood sample, associated with the preeclampsia phenotype than in a sample, e.g. blood sample, not associated with the preeclampsia phenotype. In other instances, a reduced level of the biomarker at the gestational age of sampling is associated with the preeclampsia phenotype. For example, the concentration of marker in a sample, e.g. blood sample, may be 1.5-fold, 2-fold, 2.5-fold, 3-fold, 4-fold, 5-fold, 7.5-fold, 10-fold, or greater less concentrated in a sample, e.g. blood sample, associated with the preeclampsia phenotype than in a sample, e.g. blood sample, not associated with the preeclampsia phenotype or the concentration of the biomarker in a sample, e.g. blood sample, may be 10%, 20%, 30%, 40%, 50% or more less concentrated in a sample, e.g.

blood sample, associated with the preeclampsia phenotype than in a sample, e.g. blood sample, not associated with the preeclampsia phenotype.

Preeclampsia biomarkers used in the method of the present invention include PTX3, sFlt1, ADAM12 and sENG. The level of at least three, three, or four are measured in the sample, preferably a blood sample, from the patient. The level of three or more or four biomarkers are measured in the sample, preferably a blood sample, from the patient. The levels of the biomarkers in the sample, e.g. blood sample, find use in providing a preeclampsia assessment, for example making a preeclampsia exclusion, prognosis, monitoring and/or treatment. Preeclampsia biomarkers may be measured in in vitro cell culture.

In one embodiment of the invention, the method comprises measuring the level of at least three biomarkers. The method may comprise measuring the level of at least three biomarkers, wherein the at least three biomarkers are selected from PTX3, sFlt1 and ADAM12 and optionally sENG. The method may comprise measuring the level of at least three biomarkers, wherein the at least three biomarkers are selected from PXT3, ADAM12, sENG and optionally sFlt1. In a preferred embodiment of the invention, the method comprises measuring the level of three biomarkers wherein the three biomarkers are PTX3, sFlt1 and ADAM12. In another preferred embodiment of the invention, the method comprises measuring the level of three biomarkers wherein the three biomarkers are PXT3, ADAM12, sENG. In a further preferred embodiment of the invention, the method comprises measuring the level of four biomarkers, wherein the four biomarkers are PTX3, sFlt1, ADAM12 and sENG.

### Method

The level(s) of preeclampsia marker(s) in the biological sample, e.g. blood sample, from an individual are measured or evaluated. The terms “evaluating”, “assaying”, “measuring”, “assessing,” and “determining” are used interchangeably to refer to any form of measurement, including determining if an element is present or not, and including both quantitative and qualitative determinations. Evaluating may be relative or absolute. The level of one or more preeclampsia markers in the subject sample, e.g. blood sample, may be measured or evaluated by any convenient method. For example, such methods may include biochemical assay methods, immunoassay methods, mass spectrometry analysis methods, or chromatography methods, or combinations thereof. The term “immunoassay” generally refers to methods for detecting one or more molecules or analytes of interest in a sample, e.g. blood sample, wherein specificity of an immunoassay for the molecule(s) or analyte(s) of interest is conferred by specific binding between a specific-binding agent, commonly an antibody, and the molecule(s) or analyte(s) of interest. For example, preeclampsia biomarker levels may be detected using any immunoassay-based technology, multiplex platform or other microsphere-based platform, ELISA, RIA, EIA, EMIT, FIA, FPFIA, TRFIA, CLIA, LIA or LIPs. Preeclampsia biomarker levels may be detected using mass spectrometry, proteomic arrays, flow cytometry, western blotting or immunohistochemistry.

Preferably the immunoassay-based technology used in the present invention is a multiplex platform e.g. a microsphere-based platform. More preferably, the immunoassay-based technology used in the present invention is a multiplex platform that is a microsphere-based platform e.g. xMAP (sometimes referred to as xMAP technology e.g. as supplied by Luminex). According to the present invention the microsphere-based platform used in the present invention is xMAP technology.

### Clinical Cofactors

In some embodiments of the invention, the methods of preeclampsia assessment, e.g. prognosing preeclampsia, monitoring the course of pregnancy, may comprise additional assessment(s) that are employed in conjunction with the biomarker measurement. In particular, the subject methods may further comprise measuring one or more clinical cofactors associated with preeclampsia. One such clinical cofactor is gestational age at blood sampling or blood draw. Another is parity e.g. nulliparity or multiparity at blood sampling or blood draw. A further clinical cofactor is the placental or fetal genotype of the rs4769613 T/C single nucleotide polymorphism e.g. TT-, CC- or TC- genotype. In an embodiment of the invention, the method comprises measuring at least one, at least two or three clinical cofactors. In an embodiment of the invention, the method comprises measuring at least two clinical cofactors that is gestational age and parity. In another embodiment of the invention, the method comprises measuring at least two clinical cofactors selected from gestational age and the placental or fetal genotype of the rs4769613 T/C single nucleotide polymorphism. In a further embodiment of the invention, the method comprises measuring three clinical cofactors selected from gestational age, parity and the placental or fetal genotype of the rs4769613 T/C single nucleotide polymorphism.

A subject may be assessed for one or more clinical cofactors at about week 10 or more of gestation, e.g. week 11, 12, 13 or 14 of gestation, wherein the clinical cofactors are used in combination with the marker level representation to provide a preeclampsia prognosis, to monitor the preeclampsia. In some instances, the clinical cofactors may be measured prior to obtaining the preeclampsia biomarker level representation. In some instances, the clinical cofactors may be measured after obtaining the preeclampsia biomarker level representation. In some embodiments of the invention, the clinical cofactors are measured at the same time as the sample, e.g. blood sample, drawn and biomarkers measured from the subject. In some other embodiments of the invention, the clinical cofactors may be measured +/−1, 2, 3 or 4 days from the sampling of the subject.

### Prediction Value

The measurements of the preeclampsia biomarkers and optionally the clinical cofactors may be analysed collectively to arrive at a single preeclampsia prediction value. Prediction value means a single metric value that represents the weighted levels of each of the preeclampsia biomarkers and optionally clinical cofactors measured. As such, the method comprises detecting the level of biomarkers in the sample, e.g. blood sample, and optionally determining at least two clinical cofactors and calculating a prediction value based on the levels of the preeclampsia biomarkers and optionally the at least two clinical cofactors. The prediction value is compared to a cut off or threshold value and a prediction of whether the subject will develop or has preeclampsia will be made based on that comparison. The prediction value represents a prediction as to whether the subject will develop or not preeclampsia within a given time period, e.g. at or after gestational week 33+0 (day 231) until delivery. The prediction value calculated at the 10^{th} 11^{th}, 12^{th}, 13^{th} or 14^{th} gestational week may predict whether the subject will develop or will not develop preeclampsia with an onset at or after 20^{th}, 21^{st}, 22^{nd}, 23rd, 24^{th}, 25^{th}, 26^{th}, 27^{th} 28^{th}, 29^{th}, 30^{th}, 31^{st}, 32^{nd}, 33rd, 34^{th}, 35^{th}, 36^{th}, 37^{th}, 38^{th}, 39^{th}, 40^{th}, 41^{st} or 42^{nd} gestational week. Preferably, the prediction value indicates whether the subject will develop or will not develop preeclampsia at or after gestational week 33+0 (day 231) until delivery.

In an embodiment of the invention, the method comprises generating a prediction value based on the levels of the at least three biomarkers in the sample, e.g. blood sample, from the subject and optionally based on the at least two clinical cofactors and generating a prediction value that is compared to a threshold value. In the case where the prediction value, calculated using the serum biomarker and clinical data of the patient at the time of blood sampling, is equal to, greater than or larger than the threshold value, the subject is predicted to have a risk of developing PE, i.e. PE is ruled-in. In the case where the prediction value, calculated using the serum biomarker and clinical data of the patient at the time of blood sampling, is less than or lower than the threshold value, the risk to PE is predicted to be zero, i.e. ruled out. The threshold value may be between 0.1 and 0.5. In some embodiments of the invention, the threshold value is 0.412+0.005. In other embodiments of the invention the threshold value is 0.243+0.005. In yet further embodiments of the invention the threshold value is 0.356+0.005. The method of the present invention predicts whether the subject has, will or will not develop preeclampsia during the ongoing pregnancy.

In a further embodiment of the invention, a high PTX3, high ADAM12 and a high sFlt1 level, and if included in the calculation of the prediction value, a high sENG, or a high PTX3, high ADAM12 and a high sENG level, and if included in the calculation of the prediction value, a high sFlt1 as compared with control indicates an increased susceptibility of the subject developing preeclampsia or increased probability that the subject will develop preeclampsia and if included in the calculation of prediction value, a low gestational age, nulliparity, placental or fetal CT- and CC-genotypes of the rs4769613 T/C single nucleotide polymorphism indicates an increased probability of the subject to develop preeclampsia or increased probability that the subject will develop preeclampsia. In an embodiment of the present invention, the prediction value is based on gestational age and parity, gestational age and the placental or fetal genotype of the rs4769613 T/C single nucleotide polymorphism or gestational age, parity and the placental or fetal genotype of the rs4769613 T/C single nucleotide polymorphism as clinical cofactors.

The prediction value is typically compared to a threshold value. Thus, the prediction of whether the subject will develop PE is by reference to a threshold value. When the prediction value is compared to a threshold value, it indicates whether the subject will develop or not PE within a time period. The indication that the subject will develop PE typically means that the subject will develop PE at or during 5, 6, 7, 8 or 9 gestational months. The person will typically develop PE at or after 20, 21, 22, 23, 24, 25, 26, 27, 28, 29, 30, 31, 32, 33, 34, 35, 36, 37, 38, 39, 40, 41 or 42 weeks' gestation. In a preferred embodiment, the subject will develop PE at or after week 33+0 (day 231) until delivery. If the comparison of the prediction value to the threshold indicates that the subject will not develop PE, this means that the subject will not develop PE at or after 20, 21, 22, 23, 24, 25, 26, 27, 28, 29, 30, 31, 32, 33, 34, 35, 36, 37, 38, 39, 40, 41, or 42 weeks' gestation or at or after week 33+0 (day 231) until delivery. This is informative to the patient management as no specific preventive treatment and specific follow-up monitoring is needed until delivery.

A prediction value (p(i)) for a patient sample, e.g. blood sample, may be calculated by an algorithm for using biomarker concentrations.

The algorithm may be according to the following formula:

$$p ( i ) = 1 / ( 1 + {e}^{∧} ( - a - b \times log 2 ( [ PTX 3 ] ) - c \times log 2 ( [ sFlt 1 ] ) - d \times log 2 ( [ sENG ] ) - e \times log 2 ( [ ADAM 12 ] ) - f \times parity - g \times gest . age - h \times placental or fetal genotype of rs 4769613 SNP ) )$$

wherein a, b, d, e, f and h are positive numbers and c and g are negative numbers;
or one or more of f, g and h are 0.

The coefficient values may be as follows:

$$a = 8.7 \pm 10 \% \\ b = 2.1 \pm 10 \% \\ c = - 3.9 \pm 10 \% \\ d = 1.9 \pm 10 \% \\ e = 2.7 \pm 10 \% \\ f = 20.8 \pm 10 \% \\ g = - 0.1 \pm 10 \% \\ h = 2.5 \pm 10 \%$$

The threshold value may be 0.412+0.005, and a p(i) value equal to or above the threshold value indicates the subject will develop preeclampsia at or after gestational week 33+0 (day 231) until delivery or a p(i) value below the threshold value indicates the subject will not develop preeclampsia at or after gestational week 33+0 (day 231) until delivery.

The algorithm may be according to the following formula:

p(i)=1/(1+e\hat{ }(−a−b×log 2([PTX3])−c×log 2([sFlt1])−d×log 2([sENG])−e×log 2([ADAM12])−f×parity−g×gest.age))

wherein a, b, e and f are positive numbers, c, and g are negative numbers and d is 0; or one or more of f and g are 0.

The coefficients values are as following:

$$a = 15.9 \pm 10 \% \\ b = 1.9 \pm 10 \% \\ c = - 1.8 \pm 10 \% \\ d = 0 \\ e = 1.7 \pm 10 \% \\ f = 4.1 \pm 10 \% \\ g = - 0.1 \pm 10 \%$$

The threshold value may be 0.243+0.005 and a p(i) value above the threshold value indicates the subject will develop preeclampsia at or after gestational week 33+0 (day 231) until delivery or a p(i) value below the threshold value indicates the subject will not develop preeclampsia at or after gestational week 33+0 (day 231) until delivery.

The algorithm may be according to the following formula:

$$p ( i ) = 1 / ( 1 + {e}^{∧} ( - a - b \times log 2 ( [ PTX 3 ] ) - c \times log 2 ( [ sFlt 1 ] ) - d \times log 2 ( [ sENG ] ) - e \times log 2 ( [ ADAM 12 ] ) - g \times gest . age - h \times placental or fetal genotype of rs 4769613 SNP ) )$$

wherein a, b, d, e and h are positive numbers and c and g are negative numbers;
or one or more of g and h are 0.

The coefficients values may be as follows:

$$a = 11.2 \pm 10 \% \\ b = 2.1 \pm 10 \% \\ c = - 3.5 \pm 10 \% \\ d = 2.8 \pm 10 \% \\ e = 3.8 \pm 10 \% \\ g = - 0.1 \pm 10 \% \\ h = 3.3 \pm 10 \%$$

The threshold value may be 0.356+0.005, and a p(i) value above the threshold value indicates the subject will develop preeclampsia at or after gestational week 33+0 (day 231) until delivery or a p(i) value below the threshold value indicates the subject will not develop preeclampsia at or after gestational week 33+0 (day 231) until delivery.

In the calculation of p(i):
[PTX3] is the concentration of PTX3 in the sample in [pg/mL]
[sFlt1] is the concentration of sFlt1 in the sample in [pg/mL]
[sENG] is the concentration of sENG in the sample in [ng/ml]
[ADAM12] is the concentration of ADAM12 in the sample in [ng/ml] gest age is the gestational age in days at sampling parity is whether the subject has had no previous pregnancies (nulliparity) or one or more pregnancies (multiparity). Parity is binary, nulliparity=1, multiparity=0. Thus, nulliparity increases the risk that the subject will develop preeclampsia. Multiparity decreases the risk that the subject will develop preeclampsia.

Placental or fetal genotype rs4769613 is the placental or fetal genotype of the rs4769613 T/C single nucleotide polymorphism e.g. CC, CT or TT. CC=2, CT=1 and TT=0

The caret (\hat{ }) refers to the exponentiation operator.

In further embodiments, alternative algorithms combining in an additive manner three or more serum biomarkers comprising [PTX3], [ADAM12] and [sFlt1], [PTX3], [ADAM12] and [sENG] or [PTX3], [ADAM12], [sFlt1] and [sENG] with or without clinical cofactors can be applied.

### EXAMPLES

### Materials and Methods

Luminex xMAP® technology

xMAP® suspension array technology is based on polystyrene beads with a diameter of 5.6 μm that are internally dyed with various ratios of two spectrally distinct fluorophores (Luminex® Corporation, Austin, TX, USA). As a result, an array of up to 500 different bead sets with specific absorption spectra is created. Various biological molecules, such as individual oligonucleotide probes, proteins or antibodies, can be coupled to alternative sets of beads. These sets are combined to a suspension array and due to their unique absorption spectra, it is possible to measure simultaneously up to 500 different probes in a single multiplex reaction. The technology is capable of performing both protein- and nucleic acid-based analyses, enabling both quantitative protein assays and qualitative DNA-based detection assays.

Luminex® technology: https://www.luminexcorp.com/research/our-technology/xmap-technology/Reagents for the Luminex® sandwich immunoassays

Development of the Luminex® xMAP based multiplexed assay for the measurement of maternal serum biomarkers for the prognosis of preeclampsia has been described in detail in Ratnik et al 2020. The developed 6PLEX assay enables to simultaneously measure serum concentrations of ADAM12, sENG, leptin, PIGF, sFlt-1, and PTX3 biomarkers. Luminex® magnetic microspheres (#MC100) and antibody coupling kit for covalent linking of antibodies and microspheres (Antibody Coupling Kit, #40-50016) were purchased directly from Luminex® Corporation (Austin TX, USA). Capture and detection antibodies, and reference proteins were purchased from R&D Systems (Minneapolis, MN, USA). The best performing antibody combinations are presented in Ratnik et al 2020. Antibodies were covalently linked to the surface of the fluorescently labeled Luminex® microspheres according to the manufacturer's protocol and kept in dark at +4° C. until use. Stock solutions of each reference protein were prepared according to respective manufacturer's instruction. Stock solutions were further diluted in 1% BSA (#810685; Merck KGaA, Darmstadt, Germany) to prepare the 10X Standard-8 solutions of either individual or multiplexed reference proteins, corresponding to 10-fold higher concentration of the maximum expected value of each analyte (Ratnik et al 2010). Prior to each immunoassay experiment, fresh working solutions the Standard-8 and its two-fold serial dilutions (Standard-7 and subsequent dilutions representing decreasing concentrations) were prepared in General Assay Diluent (GAD; #620; ImmunoChemistry Technologies LLC, Minnesota, USA). GAD represents a mammalian protein-based immunoassay additive and it served as a blank measurement (Standard-1) for each analyzed biomarker in all experiments. In immunoassay experiments, the Serum Matrix (S1-100ML, Human Serum; Merck KGaA, Darmstadt, Germany), the Serum Matrix with the spiked proteins and tested serum samples were diluted in GAD. All reagents were kept at −80° C.

Luminex® sandwich immunoassay protocol

All incubations during the immunoassay protocol were carried out at room temperature (RT) using a microplate shaker with rotational movements (speed set to 500 rpm; #4625, Barnstead Lab-Line, MA, USA). At the end of each incubation step, plates were placed on the Luminex® Magnetic Plate Separator (#CN-0269-01) for 1 min to bring all magnetic microspheres to the bottom of each well, followed by supernatant aspiration. All washing steps between incubations used 100 L of WB per cell.

Prior to the immunoassay, 96-well round bottom microplates (#734-1642 Corning, NY, USA) were treated for 10 min with Blocking Buffer (BB; 100 uL per well; 1% BSA, 0.02% Tween-20 in PBS at pH 7.4). After removal of BB, capture antibody coupled microbead solution prepared in WB (50 μL per well containing 2,500 beads of each analyte) and tested samples (′Standards8-1′ or sera diluted in GAD; 50 μL/well) were pipetted to microplates. The mixture was incubated together for 2 h, followed by a washing step. Next, a biotinylated monoclonal or polyclonal detection antibody (or mixture of antibodies for multiplex assays; 100 μL/well) diluted in WB was incubated for 1 h with a subsequent washing step. For the labeling of the immunoassay with a fluorescent reporter, incubation with streptavidin-phycoerythrin conjugate (100 μL of 1 ug/mL solution in WB; #PZPJ39S, Europa Bioproducts Ltd, Cambridge, UK) was applied for 30 min, followed by two rounds of washings. Finally, microbeads were resuspended in 75 μL of WB, a minimum of 50 biomarker-specific beads were collected from each well and analysed on Luminex® MAGPIX analyzer (Luminex®) Corporation, Austin TX, USA) using weighted 5-parameter logistic model implemented in Luminex® xPONENT 4.1 software (Luminex® Corporation, Austin TX, USA).

Analytical accuracy, Serum Matrix Coefficient, limits of detection (LoD), inter- and intraassay variability of each biomarker measurement is presented in details in Ratnik et al 2020.

Happy Pregnancy cohort of pregnant women and clinical study material in the current study

The ‘Happy Pregnancy’ study (full name: ‘Development of novel non-invasive biomarkers for fertility and healthy pregnancy’, 2013-2015) recruited 2,334 unselected pregnant women at their first antenatal visit at the Women Clinic of Tartu University Hospital, Estonia. The study was approved by the Ethics Review Committee of Human Research of the University of Tartu, Estonia (permissions no 221/T-6, 17.12.2012; 286/M-18, 31.01.2018). Informed consent was obtained from every subject at first antenatal visit. All pregnancies were monitored until delivery based on the recommendations of the national guidelines for antenatal care. For every participant, longitudinal anthropometric, epidemiological (three questionnaires across gestation), clinical data and biological material throughout the pregnancy and at the delivery were collected. Serum samples for research purposes were collected from the study participants in parallel with blood sampling for routine clinical tests and transferred to and stored at −80ºC. At every clinical visit, all study participants had been monitored for their weight gain, arterial blood pressure dynamics, symptoms of proteinuria and signs of PE. The data about the further course and pregnancy outcome including fetal parameters were obtained from medical documentation.

In the current study, 34 serum samples (14 PE, 20 non-PE) drawn between 70-98 gestational days (from 10+0 until 14+0 gestational weeks, inclusive) representing 31 pregnancies from pregnant women (age 18-39 years) with spontaneously conceived single pregnancy, were analysed (Table 2). In three cases, two blood samples had been drawn during this period. Individuals with multiple pregnancy, pre-existing renal disease or anti-phospholipid syndrome were excluded. During their first antenatal visit, all women were normotensive, including two subjects with medical history of antihypertensive treatment. At the first trimester blood draw, all the women were asymptomatic in regards to the signs of PE. Two of the cases developed early onset PE (<34 gestational weeks) and 12 late onset PE.

Single measurements of increased blood pressure (systolic blood pressure, SBP ≥140 mmHg, diastolic blood pressure, DBP >90 mmHg) or signs of impaired renal function (e.g. proteinuria) at routine antenatal visits were considered as clinical signs alerting to elevated risk for PE. Hypertension was confirmed when the abnormal measurements were obtained in sitting position in two occasions at least 4 hours apart while the patient has been resting for at least 15 minutes.

Diagnosis of PE was based on the criteria recommended by the International Society for the Study of Hypertension in Pregnancy (ISSHP) and the American College of Obstetricians and Gynecologists (ACOG, 2013). PE was assigned at the onset of both hypertension (SBP ≥140 mmHg; DBP ≥90 mmHg) and proteinuria (2+protein or greater on dipstick urinalysis, ≥300 mg of protein per 24-h urine collection). Alternative to increased urinary protein, other relevant clinical symptoms were considered, such as headache resistant to analgesics or visual disturbances, epigastric pain, severe edema, and oliguria after 20 gestational weeks.

During the ongoing/index pregnancy 13 women had eventually developed PE (age 26.4+3.7 years; pre-pregnancy BMI 24.1+4.3; nulliparity 92.9%; male newborn 50.0%) and 18 gestations proceeded until delivery without PE (age 26.9+4.4 years; pre-pregnancy BMI 25.8+4.9; nulliparity 40.0%; male newborn 55.0%). Further details of the Happy Pregnancy study cohort, clinical characteristics of the analyzed study group and diagnostic criteria for PE are provided in Kikas et al 2020.

Biomarker measurements in clinical serum samples with Luminex® 6PLEX immunoassay

All blood samples were collected into Becton Dickinson Vacutainer@ SST™ Serum Separation Tubes containing spray-coated silica and a polymer gel (Becton Dickinson Company, Franklin Lake, NJ, USA). Serum was separated in the service laboratory (United Laboratories, Tartu University Hospital) using routine procedures according to manufacturer's instructions (centrifugation at 1,800 g for 10 min at RT). Serum samples were kept at −80° C. with no thawing for maximum 1.5 years before further aliquoting and subsequent analysis.

Prior to the immunoassay applications, all 34 clinical serum samples selected for the current study were thawed on ice and distributed to smaller (200 μl) aliquots, to be immediately returned to −80° C. All analyzed samples were aliquoted within the same week. One aliquot of each clinical serum sample was utilized in the analysis of biomarker concentrations with the developed Luminex® 6PLEX immunoassay (Ratnik et al 2020) combining the measurements of PTX3, sFlt1, ADAM12 and sENG (included in the 1^{st} trimester PE prognosis model), as well as leptin and PIGF (excluded from the model). All 34 clinical serum samples (1:20 dilutions in GAD) were measured in duplicate during the same experiment and the mean of the two parallel MFI (mean fluorescence intensity) values was utilized in subsequent calculations. The concentrations of the analytes were calculated based on the dilution series of the reference proteins kept at −80° C. A mixture of 10× concentrated reference proteins contained sFlt1 100 ng/ml, PIGF 5 ng/ml, sENG 100 ng/ml, ADAM12 2000 ng/mL, leptin 100 ng/ml and PTX3 100 ng/ML (Ratnik et al 2020).

Genotyping the placental or fetal genetic risk factor for the preeclampsia development The SNPs rs4769613 T/C, localized near the FLT1 gene, represents a placental or fetal genetic risk factor for PE (McGinnis 2017; Kikas 2020). This genetic marker was genotyped using pre-made Taqman genotyping assays according to manufacturer's protocol (Applied Biosystems, Foster City, USA; Assay ID: C_32231378_10, C 1445411_10). Placental or fetal tissues were available for genotyping for 12 PE and 19 non-PE cases.

### Statistical Analysis

Statistical analyses were implemented using STATA/SE 13.0 (StataCorp LLC; Texas, USA) or the R3.3.3 language and environment (Free Software Foundation, Boston, MA, USA, http://www.r-project.org). Statistical differences in biomarker levels among clinical subgroups were assessed using Mann-Whitney rank sum test. P-value <0.05 was considered statistically significant.

Logistic regression models (glm) implemented in R were applied to investigate associations between biomarker measurements and clinical onset of PE during the ongoing/index pregnancy. All biomarker values were centred and scaled before modelling for data normalization and standardization. Before implementing the glm package a first automated computational pre-filtration was performed by using CARET package with training method LOOCV (leave one out cross validation) and modelling with stepAIC (generalized linear model with stepwise feature selection). This pre-filtration method (referred as LOOCV+stepAIC approach) allows to select and start with the statistically most significant prognosis model (model 4A, Table 3). Pre-filtration was carried out by using following input variables: measured concentrations of ADAM12, leptin, Pentraxin3, sENG, sFlt-1, PIGF and maternal characteristics of blood sampling time in gestational days, weight at blood draw and parity as binary variable. The best predicted model by the LOOCV+stepAIC approach (measurements of PTX3, ADAM12, sFlt-1 adjusted for gestational age and parity; model 4A, Table 3) was developed further by alternatively replacing sFlt-1 with sENG or considering them both in the model (model 4B-C). Additionally, statistical models were built combining parameter combinations from model 4A-C with the placental or fetal genotypes of the SNP rs4769613 T/C either by replacing parity with the SNP data (models 5A-C) or considering them both (models 6A-C).

The predictive power of the best models was assessed using the area under the curve (AUC). For every model, a corresponding formula was developed along with the calculated threshold value for PE prediction, and coefficients for the included biomarkers and clinical characteristics.

The best performing prediction models were retrospectively applied to the analyzed clinical cases to estimate the rate of false-positive and false-negative PE predictions, and to assess case-by-case the clinical scenarios for biased test performance. The function ‘predict’ in the package ‘stats’ was used to obtain individual predictions from a fitted glm model objects (for individual cases). Application of the formula generates the PE prediction value (p(i)) for the analyzed patient during the ongoing/index pregnancy until term. The (p(i)) equal or superior to a threshold value indicates that the subject will develop PE or has PE, whereas the (p(i)) inferior to a threshold value rules out PE development.

### Example 1

Biomarker Levels in 10-14^{th} Gestational Week, Measured with Luminex® 6PLEX Assay Biomarker Data

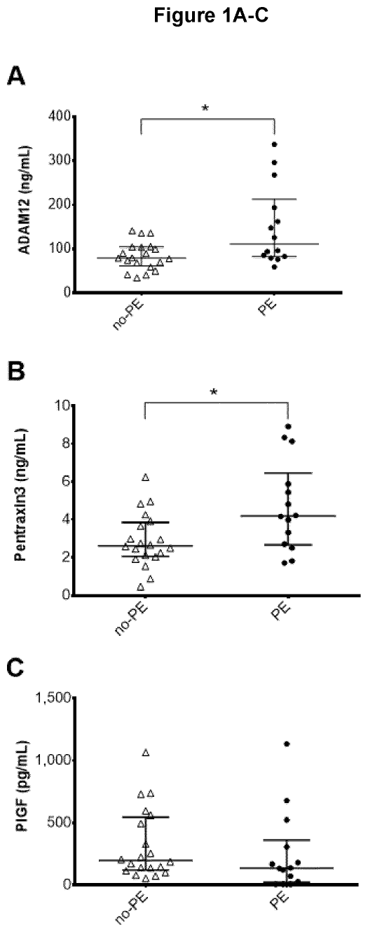

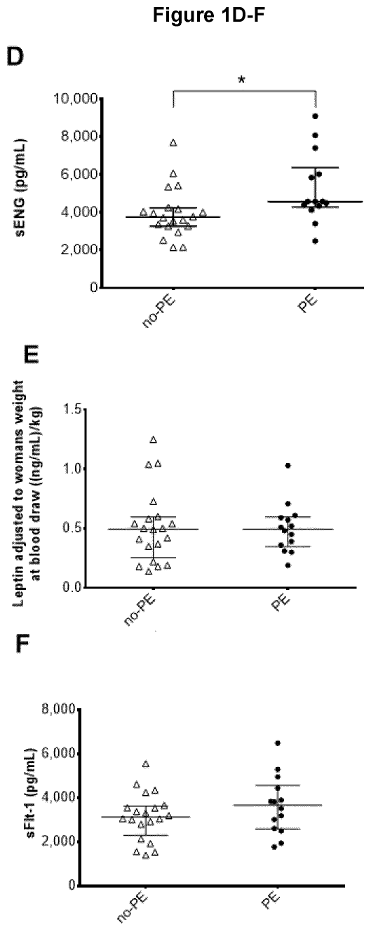

Luminex® 6PLEX assay was implemented to analyze serum samples drawn during 10-14^{th} gestational weeks from women proceeding with an uncomplicated pregnancy until term and patients who developed preeclampsia (PE) during 231-281 (median 257) gestational days but were asymptomatic at the blood draw scheduled 142-189 (median 164.5) gestational days before PE diagnosis. First trimester serum measurements of ADAM12 (Mann-Whitney U-test, p=0.017), PTX3 (P=0.027) and sENG (P-8.5×10⁻³) were significantly increased in cases with later PE (FIG. 1). Although the differences in sFlt-1 and PIGF serum between the groups did not reach statistical significance, the measurements showed a trend for increased sFlt-1 (median 3660 vs 3110 pg/mL) and decreased PIGF (136.1 vs 196.4 pg/mL) in patients developing PE during this pregnancy. There was a statistically significant positive correlation between sFlt-1 and sENG measurements (Spearman's R=0.55; P=7.3×10−4;

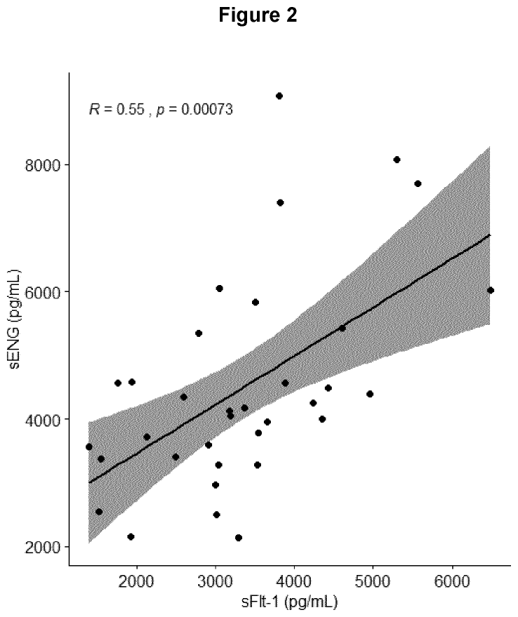

FIG. 2). First trimester serum leptin measurements (adjusted for maternal weight) did not differ between women, who developed PE during the ongoing/index pregnancy and the control cases.

### Example 2

PE prognosis models combining serum biomarkers adjusted for gestational age and parity

The PE prognosis models were developed using prospective measurements of 34 serum samples (PE, n=20, non-PE, n=14) drawn at 70-98^{th} gestational days (Table 2) from cases who developed PE (n=14; 2 early-onset, 12 late-onset cases) and from healthy pregnant women (n=20). Prognostic models were built using three settings (Table 3):
Models 4A-C: biomarker measurements in combination with gestational age and parity
Models 5A-C: biomarker measurements in combination with gestational age and placental or fetal genotypes of SNP rs4769613 T/C, a genetic risk factor for PE
Models 6A-C: biomarker measurements in combination with gestational age, parity and placental or fetal genotypes of SNP rs4769613 T/C

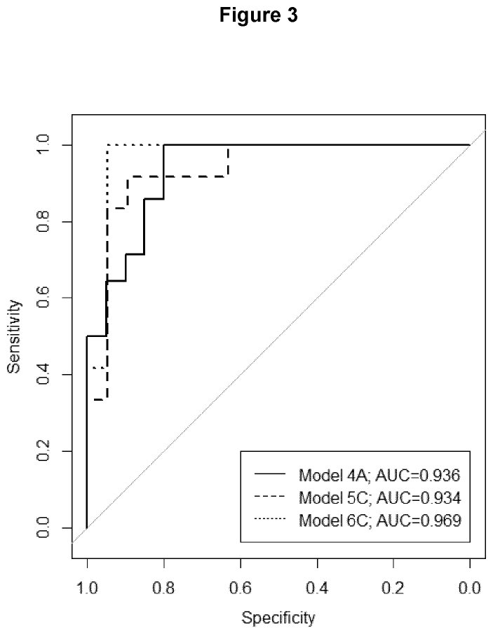

When applying a computational pre-filtration LOOCV+stepAIC approach for the selection of statistically the most significant prognosis model, marker combination 4A (measurements of PTX3, ADAM12, sFlt-1 adjusted for gestational age and parity) appeared as the best model (AUC=0.936 [0.843-0.993]; Table 4, FIG. 3). The model provided 100% [96% CI, 92.9-100] sensitivity in detecting all future PE cases, irrespective of the gestational age of PE diagnosis (between 231 and 281 gestational days). It reached 80% [95% CI 65-100] of prognosis specificity. Four of the 34 measured sera (11.8%) resulted in false positive (FP) predictions with estimated accuracy of the model 88.2% [95% CI, 73.4-95.3]). One FP case (Case C, Table 5) was characterized by maternal pre-existing endocrine disturbances hypothyroidism and polycystic ovary syndrome (PCOS), and developed gestational hypertension from 38^{th} week onward (Table 5). All four cases delivered at term (between 40+0 and 40+6 gestational weeks) and at an appropriate weight for the gestational age of the newborns (3.5-4.2 kg).

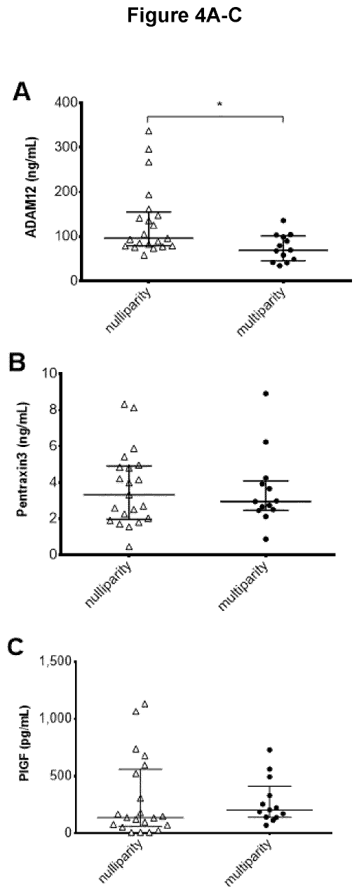

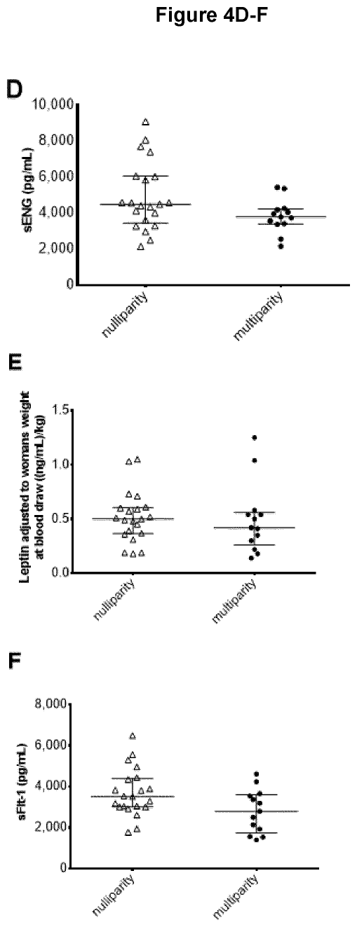

Consistent with the redundancy of the information derived from sFlt-1 and sENG serum levels (FIG. 2), when extending the model 4A by inclusion of sENG data, its prognostic properties did not improve (model 4C, Table 2). When sFlt-1 was replaced by with sENG measurements, it resulted in one additional false-positive prediction (model 4B, Table 2). PTX3 was the most important independent contributor to the model 4A, followed by parity (individual contributions for both, P<0.05), whereas ADAM12 and sFlt-1 mainly improved the specificity of PE prognosis. When combining only PTX3 measurements and parity information, the prognosis was still very good (AUC=0.886, sensitivity=99%, specificity=70%) with both markers significantly contributing to the model (PTX3, P=0.02; parity P=0.007). Information content of the PTX3 serum levels was independent from the risk to PE due to nulliparity (nulliparous vs multiparous, Mann-Whitney U-test P=0.99), whereas for ADAM12 (P=0.01), sFlt-1 (P=0.06) and sENG (P=0.08), first trimester serum levels were higher in nulliparous cases (FIG. 4).

### Example 3

PE prognosis models combining serum biomarkers adjusted for gestational age and placental or fetal genetic risk factor for PE

Alternatively, gestational age adjusted biomarker measurements were combined with the placental or fetal genotypes of the PE-risk variant rs4769613 T/C localized near FLT1 gene (data available for 12 PE and 19 non-PE cases; McGinnis 2017; Kikas 2020). The performance of the best model 5C with the genetic factor combined with PTX3, sFlt-1, sENG, ADAM12 adjusted for gestational age nearly reached the prognostic potential of model 4A (model 5A, AUC-0.934 [95% CI 0.825-1.000]; Table 4, FIG. 3). In model 5A, PTX3 and ADAM12 exhibited independent significant contributions (p<0.05). The model 5A incorporating placental or fetal genetic risk factor information exhibited increased specificity (89.5 vs 80%), but lower sensitivity (91.7 vs 100%) compared to model 4A based on biomarkers adjusted to gestational age and parity.

Model 5A prognosed correctly 27/31 analyzed cases, three FP (9.7%) and one false negative (3.2%) with estimated accuracy 87.1% [95% CI, 71.2-94.9] (Table 4). Notably, all of these cases carried the placental or fetal CC-genotype, a risk variant for PE. All FP cases progressed an uneventful pregnancy until term and delivery of a normal weight baby (3.5-4.2 kg). In two FP cases, vacuum-assisted delivery due to inadequate uterine contractions during the second stage of labor was required. The false negative (FN) case developed rapidly progressing severe early onset PE with extreme proteinuria and a C-section delivery of a newborn with intrauterine growth restriction (IUGR; Case G, Table 5). The placental histology revealed single umbilical artery that is an acknowledged risk factor for IUGR and may have also promoted PE. As the FLT1 variant rs4769613 T/C has been reported only as a risk factor for late onset PE (McGinnis et al 2017), model 5C was not able to predict PE development in this case.

### Example 4

PE prognosis models combining serum biomarkers adjusted for gestational age, parity and placental or fetal genetic risk factor for PE

Final PE prognosis models were developed incorporating serum measurements of PTX3, sFlt-1, sENG, ADAM12 adjusted for gestational age, and combined with the placental or fetal genotypes of rs4769613 T/C and parity (models 6A-C, Table 3). This parameter combination resulted in the superior PE prognosis model 6C with AUC 0.969 [95% CI 0.895-1.000], 100% sensitivity and high specificity, 94.7% [95% CI 89.5-100.0] (Table 4, FIG. 3). The model applied for the 1^{st} trimester data prognosed all future cases of PE developing in the 3rd trimester of pregnancy within the time window of 233-281 gestational days. All but case represented late-onset PE occurring only after 34^{th} gestational week.

The model resulted in two FP predictions (6.5%), one with maternal severe pre-existing endocrine disturbances and pregnancy-related hypertension (Case C, Table 5) and another (Case F) that was falsely prognosed by all developed models. The latter was characterized by late (41+4 gestational weeks) vacuum-assisted delivery due to weak contractions.

### Example 5

Formulae and utility of the preeclampsia prognosis models for 10-14^{th} gestational weeks

Taken together, the developed PE prognostic models applicable in the first trimester of pregnancy combine Luminex® 6PLEX assay measurements of maternal serum PTX3, ADAM12 with gestational age, and optionally either serum sFlt-1 or sENG measurements, parity (null—vs multiparity) or the placental or fetal genotype CC, CT or TT of the rs4769613 T/C single nucleotide polymorphism (SNP).

Although serum leptin and PIGF levels, and maternal weight had been included as the input in the statistical model building phase, these markers did not contribute to any of the PE prognosis models when measured in sera during 10-14^{th} gestational week.

The prediction value may be calculated using the following formulas and coefficients from Table 6:

$$p ( i ) = 1 / ( 1 + {e}^{∧} ( - a - b \times log 2 ( [ PTX 3 ] ) - c \times log 2 ( [ sFlt 1 ] ) - d \times log 2 ( [ sENG ] ) - e \times log 2 ( [ ADAM 12 ] ) - f \times parity - g \times gest . age )$$

$$OR$$

$$p ( i ) = 1 / ( 1 + {e}^{∧} ( - a - b \times log 2 ( [ PTX 3 ] ) - c \times log 2 ( [ sFlt 1 ] ) - d \times log 2 ( [ sENG ] ) - e \times log 2 ( [ ADAM 12 ] ) - g \times gest . age - h \times placental or fetal genotype of rs 4769613 SNP )$$

$$OR$$

$$p ( i ) = 1 / ( 1 + {e}^{∧} ( - a - b \times log 2 ( [ PTX 3 ] ) - c \times log 2 ( [ sFlt 1 ] ) - d \times log 2 ( [ sENG ] ) - e \times log 2 ( [ ADAM 12 ] ) - g \times gest . age - h \times placental or fetal genotype of rs 4769613 SNP )$$

A prediction value (p(i)) equal to, higher or greater than the threshold value, indicates that the subject will develop preeclampsia during the ongoing pregnancy.

A prediction value (p(i)) lower or less than a threshold value, indicates that the subject will not develop preeclampsia during the ongoing pregnancy, i.e. excludes the risk of developing PE.

The superior model 6C with AUC 0.969 [95% CI 0.895-1.000] included all seven parameters Luminex® 6PLEX measurements of serum PTX3, ADAM12, sFlt-1 and sENG, gestational age, parity and placental or fetal rs4769613 T/C genotypes (Tables 3-5). When applied during 10-14^{th} gestational week, the model was able to predict PE developed in third trimester without any FN cases. The fraction of FP predictions for the model 6A was 6.5% (2/31). The second best model 4A was based on Luminex® 6PLEX measurements of serum PTX3, ADAM12 and sFlt-1 adjusted for gestational age and combined with parity data (0.936 [95% CI 0.843-0.993]). Also in this model, there were no FN prognosis; the fraction of FP was 11.8% (4/34). The advantage of the model 4A is that it is straightforward to apply in a typical maternal-fetal clinical centre, whereas implementation of models 5C and 6C requires the development of diagnostic quality level genotyping of SNP rs4769613 T/C from the cell-free fetal DNA (cffDNA). cffDNA, extracted from maternal blood samples is typically applied during 10^{th}-14^{th} gestational week for the non-invasive prenatal screening (NIPS) to detect fetal chromosomal abnormalities.

This outcome of the invention (high AUC, low FP and nearly null FN) fits well to the expectations of clinical practice with the main task for early detection of risk pregnancies for timely application of preventive measures. The proportion of FP cases in the developed models was tolerable in terms of the cost and clinical burden of subsequent additional gestational monitoring of these cases.

Abbreviations: ADAM12, ADAM Metallopeptidase Domain 12; AUC, area under the curve; CI, confidence interval; PIGF, placental growth factor; ROC, receiver operating characteristics; sENG, Endoglin; sFlt1, soluble fms-like tyrosine kinase 1.

### REFERENCES

Aghababaei M, Beristain A G. The Elsevier Trophoblast Research Award Lecture: Importance of metzincin proteases in trophoblast biology and placental development: a focus on ADAM12. Placenta. 2015 April;36 Suppl 1:S11-9.
Akhter T, Wikström A K, Larsson M, Larsson A, Wikström G5, Naessen T. Serum Pentraxin 3 is associated with signs of arterial alteration in women with preeclampsia. Int J Cardiol. 2017 Aug. 15; 241:417-422.
Akolekar R, Zaragoza E, Poon L C, Pepes S, Nicolaides K H. Maternal serum placental growth factor at 11+0 to 13+6 weeks of gestation in the prediction of pre-eclampsia. Ultrasound Obstet Gynecol. 2008 November; 32(6):732-9.
Akolekar R, Minekawa R, Veduta A, Romero X C, Nicolaides K H. Maternal plasma inhibin A at 11-13 weeks of gestation in hypertensive disorders of pregnancy. Prenat Diagn. 2009 Aug;29(8):753-60.
Akolekar R, Syngelaki A, Poon L, Wright D, Nicolaides K H. Competing risks model in early screening for preeclampsia by biophysical and biochemical markers. Fetal Diagn Ther. 2013; 33(1):8-15.
Baschat A A, Magder L S, Doyle L E, et al. Prediction of preeclampsia utilizing the first trimester screening examination. AJOG. 2014; 211:514.e1-514.e7.
Brown M A, Magee L A, Kenny L C, Karumanchi S A, McCarthy F P, Saito S, Hall D R, Warren C E, Adoyi G, Ishaku S; International Society for the Study of Hypertension in Pregnancy (ISSHP). The hypertensive disorders of pregnancy: ISSHP classification, diagnosis & management recommendations for international practice. Pregnancy Hypertens. 2018; 13:291-310.
Bujold E, Roberge S, Lacasse Y, Bureau M, Audibert F, Marcoux S, Forest J C, Giguère Y. Prevention of preeclampsia and intrauterine growth restriction with aspirin started in early pregnancy: a meta-analysis. Obstet Gynecol. 2010; 116(2 Pt 1):402-14.
Colmenares-Mejía C C, Quintero-Lesmes D C, Bautista-Niño P K, Guio Mahecha E, Beltrán Avendaño M, Díaz Martínez L A, Ortiz Serrano R, Paez Leal M C, Monterrosa Castro A, Mesa
Restrepo C M, Monsalve G, Sanin-Blair E, Saldarriaga W, Luna M L, Casas J P, Serrano Díaz N. Pentraxin-3 is a candidate biomarker on the spectrum of severity from pre-eclampsia to HELLP syndrome: GenPE study. Hypertens Res. 2020 Apr 13. doi: 10.1038/s41440-020-0434-0. [Epub ahead of print]
Crovetto F, Figueras F, Triunfo S, Crispi F, Rodriguez-Sureda V, Dominguez C, Llurba E, Gratacos E. First Trimester Screening for Early and Late Preeclampsia Based on Maternal Characteristics, Biophysical Parameters, and Angiogenic Factors. Prenat Diagn 2015 Feb;35(2):183-91.
Di Lorenzo G, Ceccarello M, Cecotti V, Ronfani L, Monasta L, Vecchi Brumatti L, Montico M, D′Ottavio G. First trimester maternal serum PIGF, free B-hCG, PAPP-A, PP-13, uterine artery Doppler and maternal history for the prediction of preeclampsia. Placenta. 2012 Jun;33(6):495-501
Enquobahrie D A, Hevner K, Qiu C, Abetew D F, Sorensen T K, Williams M A. Differential Expression of HrtA1 and ADAM12 in Placentas from Preeclamptic and Normotensive Pregnancies. Reprod Syst Sex Disord. 2012; 1(3):1000110.
Garg P, Jaryal A K, Kachhawa G, Deepak K K, Kriplani A. Estimation of asymmetric dimethylarginine (ADMA), placental growth factor (PLGF) and pentraxin 3 (PTX 3) in women with preeclampsia. Pregnancy Hypertens. 2018 October; 14:245-251.
Goetzinger K R, Zhong Y, Cahill A G, Odibo L, Macones G A, Odibo A O. Efficiency of first-trimester uterine artery Doppler, a-disintegrin and metalloprotease 12, pregnancy-associated plasma protein a, and maternal characteristics in the prediction of preeclampsia. J Ultrasound Med. 2013; 32(9):1593-600.
ISUOG Practice Guidelines: role of ultrasound in screening for and follow-up of pre-eclampsia. Ultrasound Obstet Gynecol 2018; 53(1): 7-22 Hypertension in pregnancy: diagnosis and management NICE guideline Published: 25 Jun. 2019 www.nice.org.uk/guidance/ng133
Kikas T, Inno R, Ratnik K, Rull K, Laan M. 2020. C-allele of rs4769613 Near FLT1 Represents a High-Confidence Placental Risk Factor for Preeclampsia. Hypertension. 2020, https://doi.org/10.1161/HYPERTENSIONAHA.120.15346.
Kuc S, Koster M P H, Pennings J L A, Hankemeier T, Berger R, Harms A C, Dane A D, Schielen P C J I, Visser G H A, Vreeken R J. Metabolomics Profiling for Identification of Novel Potential Markers in Early Prediction of Preeclampsia. PLOS One 2014 May 29;9(5): e98540.
McGinnis R, Steinthorsdottir V, Williams N O, Thorleifsson G, Shooter S, Hjartardottir S, Bumpstead S, Stefansdottir L, Hildyard L, Sigurdsson J K, Kemp J P, Silva G B, Thomsen L C V, Jaaskelainen T, Kajantie E, Chappell S, Kalsheker N, Moffett A, Hiby S, Lee W K, Padmanabhan S, Simpson N A B, Dolby V A, Staines-Urias E, Engel S M, Haugan A, Trogstad L, Svyatova G, Zakhidova N, Najmutdinova D; FINNPEC Consortium; GOPEC Consortium, Dominiczak A F, Gjessing H K, Casas J P, Dudbridge F, Walker J J, Pipkin F B, Thorsteinsdottir
U, Geirsson R T, Lawlor D A, Iversen A C, Magnus P, Laivuori H, Stefansson K, Morgan L. Variants in the fetal genome near FLT1 are associated with risk of preeclampsia. Nat Genet. 2017 49(8):1255-1260.
Mosimann B, Amylidi-Mohr S K, Surbek D, Raio L. First trimester screening for preeclampsia a systematic review. Hypertens Pregnancy. 2020 February;39(1):1-11.
Myatt L, Clifton R G, Roberts J M, Spong C Y, Hauth J C, Varner M W, Thorp J M Jr, Mercer B M, Peaceman A M, Ramin S M, Carpenter M W, lams J D, Sciscione A, Harper M, Tolosa J E, Saade G, Sorokin Y, Anderson G D; Eunice Kennedy Shriver National Institute of Child Health and Human Development (NICHD) Maternal-Fetal Medicine Units (MFMU) Network. First-trimester prediction of preeclampsia in nulliparous women at low risk. Obstet Gynecol. 2012; 119(6):1234-42.
Myers J E, Thomas G, Tuytten R, Van Herrewege Y, Djiokep R O, Roberts C T, Kenny L C, Simpson N A, North R A, Baker P N. Mid-trimester maternal ADAM12 levels differ according to fetal gender in pregnancies complicated by preeclampsia. Reprod Sci. 2015; 22(2):235-41.
National Collaborating Centre for Women's and Children's Health (UK). Hypertension in Pregnancy: The Management of Hypertensive Disorders During Pregnancy. London: RCOG Press, 2010
O'Gorman N, Wright D, Syngelaki A, Akolekar R, Wright A, Poon L C, Nicolaides K H. Competing Risks Model in Screening for Preeclampsia by Maternal Factors and Biomarkers at 11-13 Weeks Gestation. Am J Obstet Gynecol 2016;214(1): 103.e1-103.e12.
Poon L C, Chelemen T, Granvillano O, Pandeva I, Nicolaides K H. First-trimester maternal serum a disintegrin and metalloprotease 12 (ADAM12) and adverse pregnancy outcome. Obstet Gynecol. 2008; 112(5):1082-90.
Poon L C, Karagiannis G, Leal A, Romero X C, Nicolaides K H. Hypertensive disorders in pregnancy: screening by uterine artery Doppler imaging and blood pressure at 11-13 weeks. Ultrasound Obstet Gynecol. 2009; 34(5):497-502.
Poon L C, Akolekar R, Lachmann R, Beta J, Nicolaides K H. Hypertensive disorders in pregnancy: screening by biophysical and biochemical markers at 11-13 weeks. Ultrasound Obstet Gynecol. 2010; 35(6):662-70.
Poon L C, Shennan A, Hyett J A, Kapur A, Hadar E, Divakar H, Mcauliffe F, da Silva Costa F, von Dadelszen P, Mcintyre H D, Kihara A B, Di Renzo G C, Romero R, D′Alton M, Berghella V, Nicolaides K H, Hod M. The International Federation of Gynecology and Obstetrics (FIGO) initiative on pre-eclampsia: A pragmatic guide for first-trimester screening and prevention. Int J Gynaecol Obstet. 2019;145 Suppl 1:1-33.
Ratnik K, Rull K, Hanson E, Kisand K, Laan M. Single-Tube Multimarker Assay for Estimating the Risk to Develop Preeclampsia. J Appl Lab Med. 2020 May 15:jfaa054. doi: 10.1093/jalm/jfaa054. Online ahead of print.
Redman, C W G. Current topic: pre-eclampsia and the placenta. Placenta, 1991;12: 301-308 Redman C W, Sargent I L, Staff AC. IFPA senior award lecture: making sense of pre-eclampsia two placental causes of preeclampsia? Placenta. 2014;(35 suppl):S20-S25. Rolnik D L, Wright D, Poon L C, O'Gorman N, Syngelaki A, de Paco Matallana C, Akolekar R, Cicero S, Janga D, Singh M, Molina F S, Persico N, Jani J C, Plasencia W, Papaioannou G, Tenenbaum-Gavish K, Meiri H, Gizurarson S, Maclagan K, Nicolaides K H. Aspirin versus placebo in pregnancies at high risk for preterm preeclampsia. N Engl J Med 2017; 377: 613-622.
Rovere-Querini P, Antonacci S, Dell′Antonio G, et al. 2006. Plasma and tissue expression of the long pentraxin 3 during normal pregnancy and preeclampsia. Obstet Gynecol 108: 148-155
Sober S, Reiman M, Kikas T, Rull K, Inno R, Vaas P, Teesalu P, Marti J M L, Mattila P, Laan M. Extensive shift in placental transcriptome profile in preeclampsia and placental origin of adverse pregnancy outcomes. Sci Rep. 2015; 5:13336.
Spencer K, Cowans N J, Stamatopoulou A. ADAM12s in maternal serum as a potential marker of pre-eclampsia. Prenatal Diagn 2008; 28:212-216.
Staff, A C. The two-stage placental model of preeclampsia: An update. J. Reprod. Immunol, 2019;134-135: 1-10
Stepan H, Hund M, Andraczek T. Combining Biomarkers to Predict Pregnancy Complications and Redefine Preeclampsia. The Angiogenic-Placental Syndrome. Hypertension. 2020; 75(4):918-926
Tan M Y, Wright D, Syngelaki A, et al. Comparison of diagnostic accuracy of early screening for preeclampsia by NICE guidelines and a method combining maternal factors and biomarkers: results of SPREE. Ultrasound Obstet Gynecol. 2018; 51:743-750.
Tan M Y, Syngelaki A, Poon L C, Rolnik D L, O'Gorman N, Delgado J L, Akolekar R, Konstantinidou L, Tsavdaridou M, Galeva S, Ajdacka U, Molina F S, Persico N, Jani J C, Plasencia W, Greco E, Papaioannou G, Wright A, Wright D, Nicolaides K H. Screening for pre-eclampsia by maternal factors and biomarkers at 11-13 weeks' gestation. Ultrasound Obstet Gynecol 2018; 52: 186-195.
Visintin C, Mugglestone M A, Almerie M Q, Nherera M L, James D, Walkinshaw S, Guideline Development Group. Management of Hypertensive Disorders During Pregnancy: Summary of NICE Guidance BMJ 2010; 341:c2207.
Webster K, Fishburn S, Maresh M, Findlay S C, Chappell L C, Guideline Committee. Diagnosis and Management of Hypertension in Pregnancy: Summary of Updated NICE Guidance. BMJ 2019; 366:15119.

TABLE 1
Detection rates of PE reported for 1st trimester screening algorithms for ≥2 markers^{a}

| | | Detection rate % at gestational age at | | | |
|---|---|---|---|---|---|
| | Markers used in the PE | FPR 10% | | | |
| Study | prediction algorithm | <34 w | >34 w | <37 w | PE ≥ 37 w |
| NICE 2010 | Maternal pre-pregnancy | 41 | | 39 | 34 |
| (Visintin, 2010), | factors only | | | | |
| FPR 10.2% | | | | | |

Maternal factors combined with pregnancy markers

| | | | | | |
|---|---|---|---|---|---|
| Akolekar, 2008 | UtA PI, PIGF | 89.7 | 49 | | |
| Poon, 2009 | UtA PI, MAP | 89.2 | 57 | | |
| Akolekar, 2009 | UtA PI, Inhibin A | 88.5 | 42.1 | | |
| Poon, 2010 | UtA LPI, MAP, PAPP-A, PIGF, | 92.3 | 65.6 | | |
| | Inhibin-A, Activin, TNF-R1, | | | | |
| | MMP9, PTX3, P-Selectin | | | | |
| Di Lorenzo, 2012 | UtA PI, PAPP-A, PIGF, PP13, | 75 | 31 | | |
| | hCG | | | | |
| Akolekar, 2013 | UtA PI, MAP, PIGF, PAPP-A | 96.3 | | 76.6 | |
| Kuc, 2013 | MAP, PAPP-A, PIGF, ADAM12 | 72.0 | 49.0 | | |
| Bachat, 2014 | UtA PI, MAP, PAPP-A, hCG | 55.0 | 49 | | |
| Crovetto, 2014 | UtA PI, MAP, PIGF, sFlt-1 | 91.2 | 76.4 | | |
| O'Gorman, 2017 | UtA PI, MAP, PIGF, PAPP-A | | | 75 | 48 |

^{a}Modified from Mosimann, 2020.
ADAM12, Disintegrin and metalloproteinase domain-containing protein 12;
FPR, false positive rate;
hCG, human chorionic gonadotropin;
MAP, mean arterial pressure;
MMP9, matrix metalloproteinase 9;
PAPP-A, placenta associated pregnancy protein A;
PIGF, placental growth factor;
PTX3; Pentraxin-3;
PP-13, placental protein;
sFlt-1, soluble fms-like tyrosine kinase-1;
TNF-R1, tumor necrosis factor receptor 1;
UtA LPI, uterine artery lowest PI;
UtA PI, uterine artery pulsatility index

TABLE 2
Maternal characteristics and outcome of pregnancy of the serum samples used in the study.

| | |
|---|---|
| PE n = 14 | no-PE n = 20 |

Sampling data

| Gestational age (days) | 88.7 ± 7.0 | | 88.2 ± 5.8 | |
|---|---|---|---|---|
| | 89.5 | (70-98) | 89 | (76-96) |
| Maternal age (years) | 26.4 ± 3.7 | | 26.9 ± 4.4 | |
| | 26 | (23-33) | 27 | (18-39) |
| Pre-pregnancy BMI (kg/m2) | 24.1 ± 4.3 | | 25.8 ± 4.9 | |
| | 22.9 | (18.0-32.0) | 26.0 | (16.1-34.1) |
| Maternal weight (kg) | 70.2 ± 15.1 | | 72.3 ± 15.5 | |
| | 66.5 | (48.4-95.5) | 72.3 | (49.0-103.0) |
| Mean arterial blood pressure (mmHg) | 88.4 ± 9.6 | | 85.3 ± 6.9 | |
| | 87.3 | (73.3-100.0) | 84.7 | (70.0-93.3) |

General data of the index pregnancy

| Nulliparity (%) | 13 | (92.9%) | 8 | (40.0%)* |
|---|---|---|---|---|
| Gravidity | 1.4 ± 0.9 | | 1.9 ± 0.9 | |
| | 1 | (1-4) | 2 | (1-5) |

| | | |
|---|---|---|
| Placental genotype for rs4769613 (CC/CT/TT)^{a} | 5/6/1 | 4/11/4 |

Pregnancy outcome

| Diagnosis of preeclampsia (g.d.) | 257.4 ± 15.2 | | n.a. | |
|---|---|---|---|---|
| | 257 | (231-281) | | |
| Early (<34 g. weeks)/Late onset PE (≥34 g. weeks) (n) | 2/12 | | n.a. | |
| Gestational diabetes | 0 | (0%) | 3 | (15.0%) |
| Gestational age at delivery (days) | 260.7 ± 15.6 | | 278.6 ± 12.5 | |
| | 264 | (233-281) | 283 | (236-292)* |
| Preterm delivery, <259 g. days (n, %) | 5 | (35.7%) | 1 | (5.0%)* |
| SGA newborn (n, %) | 6 | (42.9%) | 1 | (5.0%)* |
| Fetal sex (male/female) | 7/7 | | 11/9 | |
| Birth weight (grams) | 2712 ± 675 | | 3641 ± 601 | |
| | 2682 | (1664-4274) | 3729 | (1510-4198)* |

Data are given as either mean ± standard deviation and median (minimum-maximum) or number (%) for the continuous or categorical variables, respectively.
*P < 0.05; categorical variables, Chi-squared test, non-categorical variables Wilcoxon rank-sum test. BMI, body mass index; Gravidity, total number of pregnancies including index pregnancy; g. weeks, gestational weeks; IVF, in vitro fertilization; n, number of subjects; nulliparity, no previous deliveries; PE, preeclampsia Diagnosis of small-for-gestational-age (SGA) newborn was assigned at the delivery based on national guidelines (Sildver et al., 2015)
^{a}Genotype data was collected for 12 PE and 19 non-PE cases with available placental tissue; placental genotype of a single nucleotide variant rs4769613 T/C represents a risk factor for late-onset PE as it is localized in an enhancer region near FLT1, modulating gene expression (McGinnis 2017; Kikas 2020).

TABLE 3
Developed PE prognosis models and contributing variables

| Model | | | | | | | |
|---|---|---|---|---|---|---|---|
| acronym | Variables contributing to alternative models^{a} | | | | | | |
| 4A^{b} | PTX3* | sFlt-1 | | ADAM12 | parity* | | gestational days |
| 4B | PTX3* | | sENG | ADAM12 | parity* | | gestational days |
| 4C | PTX3* | sFlt-1 | sENG | ADAM12 | parity* | | gestational days |
| 5A | PTX3* | sFlt-1 | | ADAM12* | | rs4769613 T/C | gestational days |
| 5B | PTX3* | | sENG | ADAM12* | | rs4769613 T/C | gestational days |
| 5C | PTX3* | sFlt-1 | sENG | ADAM12* | | rs4769613 T/C | gestational days |
| 6A | PTX3 | sFlt-1 | | ADAM12 | parity | rs4769613 T/C | gestational days |
| 6B | PTX3 | | sENG | ADAM12 | parity | rs4769613 T/C | gestational days |
| 6C | PTX3 | sFlt-1 | sENG | ADAM12 | parity | rs4769613 T/C | gestational days |

^{a}parity was treated as a binary variable; every women was assigned as nulliparous referring to no previous deliveries, or multiparous indicating to at least one childbirth before the index pregnancy; rs4769613 T/C refers to the placental genotype of a single nucleotide variant near the FLT1 gene with T- and C-alleles
^{b}selected as statistically most significant model using the automatic computational pre-filtration approach (LOOCV + stepAIC)
*p-value < 0.05, showing a statistically significant contribution of this variable to the model ADAM12, Disintegrin and metalloproteinase domain-containing protein 12; PTX3; Pentraxin-3; sENG, soluble Endoglin; sFlt-1, soluble fms-like tyrosine kinase-1

TABLE 4
Characteristics of PE prognosis models for the biomarker
measurements 10-14th gestational weeks

| | | | | | | |
|---|---|---|---|---|---|---|
| | | false | Accuracy % | AUC | Sensitivity % | Specificity % |
| Model | n | prognosis ^{a} | [95% CI] | [95% CI] | [95% CI] | [95% CI] |

Models combining with biomarkers, gestational age and parity

| | | | | | | |
|---|---|---|---|---|---|---|
| 4A | 30/34 | 4/34 | 88.2 | 0.936 | 100.0 | 80.0 |
| | | (11.8%) | [73.4-95.3] | [0.843-0.993] | [92.9-100.0] | [65.0-100] |
| 4B | 29/34 | 5/34 | 85.29 | 0.914 | 100.0 | 80.0 |
| | | (14.7%) | [69.9-93.6] | [0.804-0.989] | [78.6-100.0] | [60.0-100.0] |
| 4C | 30/34 | 4/34 | 88.2 | 0.932 | 100.0 | 80.0 |
| | | (11.8%) | [73.4-95.2] | [0.839-0.993] | [92.9-100.0] | [65.0-100.0] |

Models combining biomarkers, gestational age and rs4769613 T/C placental genotype

| | | | | | | |
|---|---|---|---|---|---|---|
| 5A | 26/31 | 5/31 | 83.9 | 0.934 | 100.0 | 78.9 |
| | | (16.1%) | [67.4-92.9] | [0.829-1.000] | [75.0-100.0] | [68.4-100.0] |
| 5B | 25/31 | 6/31 | 80.7 | 0.930 | 91.7 | 78.9 |
| | | (19.4%) | [63.7-90.8] | [0.829-0.996] | [75.0-100.0] | [57.9-100.0] |
| 5C | 27/31 | 4/31 | 87.1 | 0.934 | 91.7 | 89.5 |
| | | (12.9%) | [71.2-94.9] | [0.825-1.000] | [83.3-100.0] | [57.9-100.0] |

Models combining biomarkers, gestational age, parity and rs4769613 T/C placental genotype

| | | | | | | |
|---|---|---|---|---|---|---|
| 6A | 29/31 | 2/31 | 93.5 | 0.969 | 100.0 | 94.7 |
| | | (6.5%) | [79.3-98.2] | [0.882-1.000] | [83.3-100.0] | [89.5-100.0] |
| 6B | 29/31 | 2/31 | 93.55 | 0.947 | 100.0 | 89.5 |
| | | (6.5%) | [79.3-98.2] | [0.851-1.000] | [83.3-100.0] | [84.2-100.0] |
| 6C | 29/31 | 2/31 | 93.55 | 0.969 | 100.0 | 94.7 |
| | | (6.5%) | [79.3-98.2] | [0.895-1.000] | [100.0-100.0] | [89.5-100.0] |

^{a} all cases were false positives, except for one false-negative case of early onset preeclampsia by model 5B and one by 5C
AUC, area under curve

TABLE 5
Pregnancies with false positive prognosis by models 4A, 5C and 6C.

| | | | | | | Newborn | | | | | | | |
|---|---|---|---|---|---|---|---|---|---|---|---|---|---|
| | Maternal data | | | | | | | Birth | Placental | | | | |
| Sample | PE/ | | age | W | Sampling | Pregnancy | Delivery | weight | weight | rs4769613 | Prognosis model | | |
| ID | No | Nulliparity | (y) | (kg) | (g.d.) | curriculum | (g.w) | (g) | (g) | T/C ^{a} | 4A | 5C | 6C |
| Case A | No | Yes | 27 | 79 | 82 | Normal | 40 + 0 | 4198 | 696 | CT | PE | No | No |
| Case B | No | Yes | 24 | 49 | 84 | Normal | 40 + 1 | 4018 | 740 | TT | PE | No | No |
| Case C [symbol] | No | Yes | 26 | 86 | 84 | GH | 40 + 6 | 3470 | 480 | CT | PE | No | PE |
| Case D [symbol] | No | Yes | 22 | 62.5 | 85 | Normal | 40 + 4 | 3506 | NA | CC | No | PE | No |
| Case E | No | No | 28 | 56 | 88 | Normal | 41 + 0 | 4042 | 550 | CC | No | PE | No |
| Case F [symbol] | No | Yes | 30 | 57 | 96 | Normal | 41 + 4 | 3776 | 796 | CC | PE | PE | PE |
| Case G [symbol] | PE | Yes | 32 | 88 | 86 | IUGR | 33 + 2 | 1664 | 372, SUA | CC | PE | No | PE |

^{a} placental genotype of a single nucleotide variant near the FLT1 gene with T- and C-alleles, a genetic risk factor for preeclampsia (McGinnis, 2017; Kikas, 2020) [symbol]
maternal pre-existing endocrine disturbances hypothyroidism and polycystic ovary syndrome (PCOS); unilateral oophorectomy a few months before getting pregnant due an ovarian cyst; isolated pregnancy-related hypertension from gestational week 38 onwards [symbol]
vacuum-assisted delivery due to weak contractions [symbol]
early onset PE with rapid progressioon and extreme proteinuria; newborn diagnosed with IUGR and hip dysplasia; placental histology revealed single umbilical artery, an acknowledged risk factor for IUGR; C-section delivery
GH, gestational hypertension;
IUGR, intrauterine growth restriction;
NA, not available;
SUA, single umbilical artery;
W, maternal weight at blood draw [symbol]
indicates data missing or illegible when filed

TABLE 6
Models and coefient values of PE prognosis models to be applied in 10-14^{th} g.weeks.

| Model/ | | | | | | | | gestational | rs4769613 |
|---|---|---|---|---|---|---|---|---|---|
| coeficient | p(i) | Intercept a | PTX3 b | sFlt-1 c | sENG d | ADAM12 e | parity f | days g | T/C h |
| 4A | 0.243 | 15.859 | 1.937 ^{a} | −1.778 | | 1.703 | 4.103 ^{a} | −0.145 | |
| 4B | 0.243 | −2.608 | 1.642 ^{a} | | −0.459 | 1.535 | 3.158 ^{a} | −0.134 | |
| 4C | 0.244 | 16.434 | 1.911 ^{a} | −1.906 | 0.363 | 1.562 | 4.124 ^{a} | −0.132 | |
| 5A | 0.182 | 5.525 | 1.710 ^{a} | −1.969 | | 3.599 ^{a} | | −0.144 | 2.227 |
| 5B | 0.208 | −14.631 | 1.146 | | 0.237 | 2.647 ^{a} | | −0.087 | 1.416 |
| 5C | 0.356 | 11.230 | 2.072 ^{a} | −3.519 | 2.799 | 3.808 ^{a} | | −0.114 | 3.333 |
| 6A | 0.308 | 2.776 | 1.871 | −2.865 | | 3.056 | 20.722 | −0.174 | 1.911 |
| 6B | 0.261 | −28.304 | 1.186 | | 0.196 | 1.549 | 19.146 | −0.054 | 1.266 |
| 6C | 0.412 | 8.665 | 2.075 | −3.907 | 1.850 | 2.718 | 20.777 | −0.136 | 2.464 |

^{a} an independent statistically ignificant contribution of the variable to the model (p-value < 0.05) rs4769613 T/C refers to the genotype of a single nucleotide variant near the FLT1 gene with T- and C-alleles; p(i) is the threshold value of the modelling, outcome equal or greater referres to high risk to PE prognosis during the index pregnancy

1. A method of prognosing preeclampsia in a subject wherein the method comprises,
measuring the level of at least three biomarkers in a sample from the subject;
optionally determining at least one clinical cofactor from the subject;
generating a prediction value, wherein the prediction value indicates whether the subject will develop or will not develop preeclampsia during the ongoing pregnancy;
the prediction value being based on the levels of the at least three biomarkers in the sample from the subject and optionally based on the at least one clinical cofactor;
wherein the at least three biomarkers are PTX3, sFlt1, ADAM12 and optionally sENG; or PTX3, ADAM12, sENG and optionally sFlt1;
the at least one optional clinical cofactor selected from gestational age, parity e.g. nulliparity or multiparity, and the placental or fetal genotype of the rs4769613 T/C single nucleotide polymorphism e.g. TT-, CC- or TC- genotype; wherein
a high PTX3, high ADAM12 and a high sFlt1 level, and if included in the calculation of the prediction value, a high sENG, as compared with control indicates an increased probability of the subject developing preeclampsia; or
a high PTX3, high ADAM12 and a high sENG level, and if included in the calculation of the prediction value, a high sFlt1, as compared with control indicates an increased probability of the subject developing preeclampsia; and
if included in the calculation of prediction value, a low gestational age, nulliparity, placental or fetal TC- and CC-genotypes of the rs4769613 T/C single nucleotide polymorphism indicates an increased probability of the subject to developing preeclampsia.

2. (canceled)

3. (canceled)

4. (canceled)

5. The method according to claim 1, wherein the prediction value is based on gestational age and parity, gestational age and the placental or fetal genotype of the rs4769613 T/C single nucleotide polymorphism or gestational age, parity and placental or fetal genotype of the rs4769613 T/C single nucleotide polymorphism as clinical cofactors.

6. The method according to claim 1, wherein the prediction value is compared to a threshold value.

7. The method according to claim 1, wherein the prediction value indicates whether the subject will develop or will not develop preeclampsia at or after gestational week 33+0 (day 231) until delivery.

8. The method according to claim 1, wherein the sample is collected at or after gestational week 10+0 (day 70) and before gestational week 14+1 (day 99).

9. The method according to claim 1, wherein the sample is a blood sample.

10. The method according to claim 1, wherein the prediction value is calculated according to a formula:
p(i)=1/(1+e\hat{ }(−a−b×log 2([PTX3])−c×log 2([sFlt1])−d×log 2([sENG])−e×log 2([ADAM12])−f×parity−g×gest.age−h×placental or fetal genotype of the rs4769613SNP))
wherein a, b, d, e, f and h are positive numbers and c and g are negative numbers;
or one or more of f, g and h are 0.

11. The method according to claim 10 wherein,
$$a = 8.7 \pm 10 \% \\ b = 2.1 \pm 10 \% \\ c = - 3.9 \pm 10 \% \\ d = 1.9 \pm 10 \% \\ e = 2.7 \pm 10 \% \\ f = 20.8 \pm 10 \% \\ g = - 0.1 \pm 10 \% \\ h = 2.5 \pm 10 \%$$

12. The method according to claim 11, wherein the prediction (p(i)) value is compared to a threshold value of 0.412±0.005.

13. The method according to claim 12, wherein a p(i) value equal to or above the threshold value indicates the subject will develop preeclampsia at or after gestational week 33+0 (day 231) until delivery, or a p(i) value below the threshold value indicates the subject will not develop preeclampsia at or after gestational week 33+0 (day 231) until delivery.

14. The method according to claim 1, wherein the prediction value is calculated according to a formula:
$$p ( i ) = 1 / ( 1 + {e}^{∧} ( - a - b \times log 2 ( [ PTX 3 ] ) - c \times log 2 ( [ sFlt 1 ] ) - d \times log 2 ( [ sENG ] ) - e \times log 2 ( [ ADAM 12 ] ) - f \times parity - g \times gest . age - h \times placental or fetal genotype of rs 4769613 SNP ) )$$
wherein a, b, e and f are positive numbers, c, and g are negative numbers and d is 0;
or one or more of f and g are 0.

15. The method according to claim 14 wherein:
$$a = 15.9 \pm 10 \% \\ b = 1.9 \pm 10 \% \\ c = - 1.8 \pm 10 \% \\ d = 0 \\ e = 1.7 \pm 10 \% \\ f = 4.1 \pm 10 \% \\ g = - 0.1 \pm 10 \%$$

16. The method according to claim 15, wherein the prediction (p(i)) value is compared to a threshold value of 0.243±0.005.

17. The method according to claim 16, wherein a p(i) value equal to or above the threshold value indicates the subject will develop preeclampsia at or after gestational week 33+0 (day 231) until delivery, or a p(i) value below the threshold value indicates the subject will not develop preeclampsia at or after gestational week 33+0 (day 231) until delivery.

18. The method according to claim 1, wherein the prediction value is calculated according to a formula:
$$p ( i ) = 1 / ( 1 + {e}^{∧} ( - a - b \times log 2 ( [ PTX 3 ] ) - c \times log 2 ( [ sFlt 1 ] ) - d \times log 2 ( [ sENG ] ) - e \times log 2 ( [ ADAM 12 ] ) - g \times gest . age - h \times placental or fetal genotype of rs 4769613 SNP ) )$$
wherein a, b, d, e and h are positive numbers and c and g are negative numbers;
or one or more of g and h are 0.

19. The method according to claim 18 wherein:
$$a = 11.2 \pm 10 \% \\ b = 2.1 \pm 10 \% \\ c = - 3.5 \pm 10 \% \\ d = 2.8 \pm 10 \% \\ e = 3.8 \pm 10 \% \\ g = - 0.1 \pm 10 \% \\ h = 3.3 \pm 10 \%$$

20. The method according to claim 19, wherein the prediction (p(i)) value is compared to a threshold value of 0.356+0.005.

21. The method according to claim 20, wherein a p(i) value equal to or above the threshold value indicates the subject will develop preeclampsia at or after gestational week 33+0 (day 231) until delivery, or a p(i) value below the threshold value indicates the subject will not develop preeclampsia at or after gestational week 33+0 (day 231) until delivery.

22. The method according to claim 1, wherein the biomarkers are measured using a multiplex platform, optionally wherein the multiplex platform is a microsphere-based platform, for example xMAP technology.

23. (canceled)

24. The method according to claim 1, wherein the subject is asymptomatic for preeclampsia.
